(19) United States
(12) Patent Application Publication
(10) Pub. No.: US 2026/0242485 A1
(43) Pub. Date: Aug. 20, 2026

(54) ANTI-CD16A ANTIBODIES AND USES THEREOF

(71) Applicant: Lepu Biopharma Co., Ltd., Shanghai (CN)

(72) Inventors: Xin Wu, Shanghai (CN); Zhihu Qu, Shanghai (CN); Yuanyuan Yang, Shanghai (CN); Feifei Cui, Shanghai (CN); Wenqing Jiang, Shanghai (CN); Lei Fang, Shanghai (CN); Hao Shen, Shanghai (CN); Handong Zheng, Shanghai (CN)

(21) Appl. No.: 19/153,770

(22) PCT Filed: Feb. 6, 2024
(86) PCT No.: PCT/CN2024/076420

§ 371 Date: Aug. 5, 2025

(30) Foreign Application Priority Data
Feb. 6, 2023 (WO) PCT/CN2023/074656

(51) Int. Cl. C07K 16/28; A61K 39/00; A61P 35/00; C07K 16/30

(52) U.S. Cl. CPC C07K 16/283; A61P 35/00; C07K 16/2866; C07K 16/303; A61K 2039/505; C07K 2317/31; C07K 2317/33; C07K 2317/52; C07K 2317/565

(57) ABSTRACT

Antibodies having highly selective specificity to the human CD16A protein, but not the CD16B protein are identified that are useful for preparing multifunctional antibodies that further have a specificity to a tumor associated antigen. Such multifunctional antibodies can activate CD16A selectively in a target-dependent manner, therefore exhibiting efficient activation and durable ADCC/ADCP function of NK cells and macrophages for treating tumors including cold tumors.

### CROSS-REFERENCED TO RELATED APPLICATIONS

This application is a U.S. National Stage of International Patent Application No. PCT/CN2024/076420 filed Feb. 6, 2024, which claims priority to International Patent Application No. PCT/CN2023/074656 filed Feb. 6, 2023, the content of which is incorporated by reference in its entirety.

### REFERENCE TO AN ELECTRONIC SEQUENCE LISTING

The content of the electronic sequence listing “75FS-347520-US.xml”, size 151,041 bytes, and date of creation of Jul. 29, 2025, is herein incorporated by reference in its entirety.

### BACKGROUND

Immunotherapy is a revolutionary therapeutic strategy in the field of cancer treatment, which had brought promising clinical benefits by harnessing the body's natural immune system to fight against tumor cells. At present, multiple immunotherapeutic strategies are being developed, including monoclonal antibodies (mAbs) targeting tumor-associated antigen (TAA) and immune checkpoint inhibitors, bi-specific and multi-specific antibodies, modified cytokines, tumor vaccines, adaptive cell therapies, mRNA drugs, etc. Among the monoclonal antibody drugs, for instance, rituximab, trastuzumab and cetuximab, targeting CD20, HER2, and EGFR, respectively, have been approved for the treatment of non-Hodgkin lymphoma (NHL), HER2 positive breast cancer and colorectal cancer/head and neck cancer. One of the major mechanisms of these drugs is that they mediate antibody-dependent cellular cytotoxicity (ADCC) and antibody-dependent cellular phagocytosis (ADCP) by natural killer cells (NK cells) and macrophages, thereby achieving clearance of the tumor cells.

As a first line defense of our body, NK cells, the key component of innate immune system, can rapidly and directly eliminate carcinoma cells, invading microbes, virus-infected cells, and external transplanted cells by NK cell activation induced cytotoxic activity and immunomodulatory functions. A balance between inhibitory and activating receptors on NK cell surface determines whether NK cells are activated or not. Among the numerous activating and inhibitory receptors expressing on the surface of NK cells, CD16A (FcγRIIIA), a member of Fc region of gamma immunoglobulins IgG (FcγRs) receptor family, is the only receptor mediating ADCC effect, which is one of the major cytotoxic mechanisms employed by NK cells to eliminate tumor cells. Mechanistically, when a specific antibody recognizes and binds to the antigen expressed on target cell, Fc fragments of the antibody will bind to CD16A on NK cells and induce crosslinking based activation of downstream signaling, triggering degranulation of cytotoxic molecules including granzyme B and perforin of NK cells, which mediate direct lysis of target cells. Meanwhile, activated NK cells release proinflammatory cytokines such as IFN-γ and TNF-α, which can recruit adaptive immune cells to participate in the elimination of target cells.

Human CD16 exists in two isoforms, CD16A and CD16B (FcγRIIIB), which share 96% sequence identity in their immunoglobulin binding regions. Due to allelic polymorphisms, CD16A has two allelic forms: CD16A-158F which is the dominant allele in about 60% of human with low affinity to IgGs, and CD16A-158V (or CD16A-176V based on the precursor sequence of SEQ ID NO:55) with high affinity to IgGs. Clinical data reveals that the high affinity CD16A-158V predicts favorable response to therapeutic IgG antibodies. Besides its expression on NK cells, CD16A also exists on the surface of monocytes and macrophages, mediating ADCP by phagocytic cells against target cells. CD16B, a GPI-anchored receptor, exhibits a quite different function due to its selective expression on neutrophils and eosinophils. It's reported that CD16B, as a decoy receptor, captures amount of IgG through Fc fragment without triggering activation of the cells, resulting in a disruptive impairment on ADCC function. There are three allelic forms of human CD16B including CD16B-NA1 (R36, N65, D82, V106), CD16B-NA2 (S36, S65, N82, I106) and CD16B-SH (D78, N65) differing in amino acids at five positions. CD32B (FcγRIIB), the only inhibitory receptor in FcγRs, is mainly expressed on B cells, macrophages, dendritic cells, neutrophils and basophils. It plays regulatory function in controlling the threshold and the extent of cell activation through Fc engagement.

Since CD16A is essential for ADCC and ADCP mediated by NK cells and macrophages, the development of CD16A targeting drugs is a promising strategy to activate NK cells and macrophages. The ideal strategy is to selectively activate both CD16A-158F and CD16A-158V allelic forms, unbiasedly without interacting with the decoy receptor CD16B and inhibitory receptor CD32B. However, the current strategy of protein engineering of Fc fragment failed to spare CD16B binding due to the high similarity in the amino acid sequences between CD16A and CD16B, although enhanced binding to both alleles of CD16A and attenuated binding to inhibitory receptor CD32B were partially achieved for some of the engineered Fc. For example, Tafasitamab (MorphoSys), an anti-CD19 mAb with engineered S239D/I332E (DE) mutation in the Fc region contributing to enhanced ADCC and ADCP function, has been approved at 2020 by EMA for second line therapy of DLBCL in combined with Lenalidomide. Obinutuzumab (Roche), a second generation of anti-CD20 mAb with complete afucosylation in the Fc region, strongly binds to both two CD16A alleles and increase ADCC effect. Nevertheless, these Fc engineering also increased the binding affinity to CD16B which may limit their clinical benefit.

An alternative strategy is to develop anti-CD16A specific antibodies for the construction of bi-specific or multi-specific fragments to activate CD16A selectively and efficiently in a target-dependent manner. These types of molecules, with one arm binding to CD16A and another arm targeting a specific antigen on target cells, can exhibit conditional and efficient activation and durable ADCC/ADCP function of NK cells and macrophages without binding to CD16B and CD32B. For example, an anti-CD16A antibody 4-LS-21 and its affinity matured version named P2C47 developed by Affimed Therapeutics showed similarly high affinity to both CD16A-158V and CD16A-158F and non-binding to CD16B and CD32B. Based on the CD16A antibody 4-LS21, two clinical assets, AFM13 (CD30-CD16A) with no Fc fragment and AFM24 (EGFR-CD16A) with a disabled Fc are currently under clinical evaluation for the treatment of relapsed peripheral T cell lymphoma, Hodgkin Lymphoma (HL) and EGFR expressing solid tumors. Recently, AFM13 was reported to have clinical responses with 100% overall response rate (ORR) in recommended phase II dose (RP2D) group when combined with NK cell therapy in CD30 positive lymphoma. Based on these previous knowledges, it is necessary to fully unlock the potential of this unique strategy in the expanded therapeutic areas by developing efficient CD16A specific antibodies and exploring appropriate components and formats of the CD16A specific bi-specific or multi-specific fragments.

The incidence of hepatocellular carcinoma (HCC) is on the rise, especially in East Asia. Since Gypican3 (GPC3) with high expressional level in liver cancer tissues is discovered in the last century, the application of GPC3 has attracted widespread attention. As a type of a heparan sulfate (HS) glycoprotein, GPC3 can interact with growth factors, extracellular matrix proteins, and adhesion molecules to regulate cell proliferation, differentiation, adhesion, and migration. Due to very limited expression in normal tissues, GPC3 is thought to be a specific TAA in HCC and the strategy of exploring GPC3 as a therapeutic target will provide a new approach for liver disease treatments. However, a randomized phase II trial (NCT01507168) evaluating a GPC3 monoclonal antibody GC33 with ADCC-enabled wild-type human IgG1 Fc region did not show clinical benefit in advanced HCC patients who had failed prior systemic therapy. Biomarker analysis revealed that high CD16A on peripheral immune cells were associated with prolonged progression free survival and overall survival. These results indicated that more efficient strategy to induce GPC3-targeted CD16A activation and the resulting ADCC and ADCP function was needed to contribute to better clinical outcomes.

### SUMMARY

The present disclosure, in various embodiments, provides antibodies and antigen-binding fragments specific to the human CD16A protein. Experimental testing shows that these newly identified antibodies can bind to the human CD16A protein potently and specifically, without interacting with the CD16B variants. Some of these antibodies also cross-react with the cynomolgus CD16 protein facilitating preclinical studies. Moreover, in vitro and in vivo studies have demonstrated that the bi-specific fragments constructed from anti-CD16A antibodies activate NK cells and mediate ADCC against target cells expressing certain antigens. Compared to other ADCC enhancing strategy, such as monoclonal antibody with engineered Fc including DE, DLE (S239D/A330L/I332E), or afucosylated Fc, anti-CD16A-based multi-specific fragments showed stronger ADCC effect. More interestingly, anti-tumor killing was further enhanced when the Fc function is intact, suggesting there is synergy between anti-CD16A and Fc-mediated effector function.

Accordingly, one embodiment of the present disclosure provides an antibody or antigen binding fragment thereof that has binding specificity to the human CD16A protein, comprising a heavy chain variable region (VH) comprising a VH CDR1, VH CDR2, and VH CDR3, and a light chain variable region (VL) comprising a VL CDR1, VL CDR2 and VL CDR3, wherein: the VH CDR1, VH CDR2, VH CDR3, VL CDR1, VL CDR2, and VL CDR3, respectively, comprise the amino acid sequences of (a) SEQ ID NO: 22, 23, 24, 37, 38 and 39; (b) SEQ ID NO: 13, 14, 15, 28, 29 and 30; (c) SEQ ID NO: 16, 17, 18, 31, 32, and 33; (d) SEQ ID NO: 19, 20, 21, 34, 35 and 36; or (e) SEQ ID NO: 25, 26, 27, 40, 41, and 42.

Also provided, in one embodiment, is a multispecific antibody, comprising: an anti-CD16A antibody or antigen-binding fragment having binding specificity to the human CD16A protein; and a second antibody or antigen-binding fragment having binding specificity to a tumor-associated antigen (TAA).

In some embodiments, the multispecific antibody further comprises an Fc fragment. In some embodiments, the anti-CD16A antibody or antigen-binding fragment and the Fc fragment have synergism when exerting effector functions. In some embodiments, the Fc fragment can bind CD64 and can not bind CD16B and/or CD32B. In some embodiments, the Fc fragment is a wildtype human IgG Fc fragment.

Methods and use for treating diseases such as cancer are also provided. In some embodiments, the cancer is prostate cancer, pancreatic cancer, leukemia, breast cancer, colorectal cancer, gastric cancer, head and neck cancer, liver cancer, esophageal cancer, cervical cancer, thyroid cancer, lung cancer, bladder cancer, kidney cancer, uterus cancer, or melanoma.

### BRIEF DESCRIPTION OF THE DRAWINGS

FIG. 1A-B show the binding capacity of anti-CD16A antibodies to human CD16A-158V-his (A) and human CD16A-158F-his (B) in an ELISA assay.

FIGS. 2A, 2B and 2C show the binding capacity of anti-CD16A antibodies to human CD16B-NA1-his, human CD16B-NA2-his and human CD16B-SH-his in an ELISA assay.

FIG. 3 shows the cross-species binding capacity of anti-CD16A antibodies to cynomolgus CD16-his in an ELISA assay.

FIGS. 4A and 4B shows the binding capacity of anti-CD16A antibodies to human CD16A-158V and CD16A-158F expressed on CHO-K1 cells measured by flow cytometry.

FIGS. 5A and 5B show the binding capacity of anti-CD16A antibodies to human CD16B-NA1 and human CD32B expressed on CHO-K1 cells measured by flow cytometry.

FIG. 6 shows cross-species the binding capacity of anti-CD16A antibodies to cynomolgus CD16 expressed on CHO-K1 cells measured by flow cytometry.

FIG. 7A-D show the binding capacity of a series of humanized anti-CD16A antibodies to human CD16A-158V, CD16A-158F, CD16B-NA1 and cynomolgus CD16 expressed on CHO-K1 cells measured by flow cytometry.

FIG. 8 shows the binding capacity of a series of humanized anti-CD16A antibodies with post-translation modification (PTM) site removal to human CD16A-158V, CD16A-158F, CD16B-NA1 and cynomolgus CD16 expressed on CHO-K1 cells measured by flow cytometry.

FIG. 9 shows the Format 1 of CD16A bi-specific fragments, containing bi-valent Fab form of antigen-binding fragments and bi-valent scFv form of anti-CD16A fragments separately on the N-terminal and the C-terminal of human IgG1 Fc fragment with or without FcγRs binding.

FIG. 10A-D show the binding capacity of various GPC3-CD16A bi-specific antibodies and anti-GPC3 mAbs against human GPC3 on GPC3+ HepG2 cells (FIG. 10A) and GPC3-SK-HEP-1 cells (FIG. 10B), human CD16A-158V (FIG. 10C) and CD16A-158F (FIG. 10D) expressed on CHO-K1 cells.

FIG. 11A-D show CD16A downstream signaling in Jurkat-NFAT cells induced by various chimeric and humanized GPC3-CD16A BsAbs against GPC3+ HepG2, Huh-7 and PLC-PRF-5 cells or GPC3− SK-HEP-1 cells.

FIG. 12A-C show primary NK cell mediated cytotoxicity toward GPC3-expressing and GPC3 negative tumor cells induced by various chimeric and humanized GPC3-CD16A BsAb.

FIG. 13A-C show primary NK cell degranulation toward GPC3-expressing and GPC3 negative tumor cells induced by GPC3-CD16A BsAb.

FIG. 14A-F show primary NK cell mediated cytokine production toward GPC3-expressing and GPC3 negative tumor cells induced by GPC3-CD16A BsAb.

FIG. 15A-C show the binding capacity of various CCR8-CD16A BsAbs and anti-hCCR8 mAbs against human CD16A-158V, CD16A-158F and CD32B expressed on CHO-K1 cells.

FIG. 16A-D show primary NK cell mediated cytotoxicity, degranulation and cytokine production toward CCR8-expressing tumor cells induced by CCR8-CD16A BsAb.

FIG. 17 shows primary NK cell mediated cytotoxicity toward CCR8-expressing tumor cells induced by various chimeric and humanized CCR8-CD16A BsAb.

FIG. 18A-B show binding capacity of in house anti-mCCR8 clone 194A1G9 to human and mouse CCR8 expressed on cells.

FIG. 19 shows ADCC signaling of 194A1G9-42F5H2 BsAb and 194A1G9 mAb.

FIG. 20 shows in vivo comparison of 194A1G9-42F5H2 BsAb and 194A1G9 mAb

FIG. 21 shows in vivo comparison of SA214G2-45H6E8 BsAb and SA214G2 mAb.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA, IgG, or IgE, respectively. The immunoglobulin subclasses (isotypes) e.g., IgG₁, IgG₂, IgG₃, IgG₄, IgGs, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-CD16A Antibodies

Through trials and errors, the instant inventors were able to identify new antibodies that can bind to the human CD16A protein potently and specifically. As shown in the experimental examples, all of the tested examples exhibited strong affinity to both the CD16A 158V polymorphic form (high-affinity) and the more prevalent 158F form (low-affinity). Interestingly, one of the newly identified antibodies, 45H6E8, also exhibited cross-reactivity to cynomolgus CD16. Also important, these new antibodies had neglectable binding to CD16B.

Yet another important finding is that these new anti-CD16A antibodies had minimum or no binding to CD32B. CD32B is the only inhibitory FcγR. The reduced interaction with FcγR, therefore, contributes to these antibodies' antitumor activities.

These newly developed antibodies were also used to create bispecific antibodies that had a second specificity to a tumor-associated antigen (TAA) GPC3 or CCR8. These bispecific antibodies were only active in triggering CD16A signaling when the coupled antigen binding portion also recognized the corresponding antigen on a target cell. These newly developed anti-CD16A antibodies, therefore, are suitable for clinical use in preventing or treating various diseases.

In accordance with one embodiment of the present disclosure, therefore, provided is an antibody or antigen binding fragment thereof that has binding specificity to the human CD16A protein, comprising a heavy chain variable region (VH) comprising a VH CDR1, VH CDR2, and VH CDR3, and a light chain variable region (VL) comprising a VL CDR1, VL CDR2 and VL CDR3, wherein: the VH CDR1, VH CDR2, VH CDR3, VL CDR1, VL CDR2, and VL CDR3, respectively, are those of antibodies shown in Tables 1, 6A and 7A. Representative CDR sequences (Kabat numbering) of these antibodies are shown in Tables 1A-B and 7B, such as those in SEQ ID NO: 22, 96, 24, 37, 38 and 39; SEQ ID NO: 22, 23, 24, 37, 38 and 39; SEQ ID NO: 13, 14, 15, 28, 29 and 30; SEQ ID NO: 16, 17, 18, 31, 32, and 33; SEQ ID NO: 19, 20, 21, 34, 35 and 36; or SEQ ID NO: 25, 26, 27, 40, 41, and 42.

In one embodiment, an antibody or antigen binding fragment thereof of the present disclosure includes the CDR regions of antibody 45H6E8, which has a VH sequence of SEQ ID NO:7 and a VL sequence of SEQ ID NO:8.

In some embodiments, the VH CDR1, VH CDR2, VH CDR3, VL CDR1, VL CDR2, and VL CDR3 have the sequences of SEQ ID NO: 22, 23, 24, 37, 38 and 39, respectively. In some embodiments, the antibody or antigen binding fragment thereof of the present disclosure includes these CDR sequences and has at least 70%, 75%, 80%, 85%, 90%, 95%, 98%, 99% or 100% sequence identity to antibody 45H6E8, or any of its humanized counterparts. In one embodiment, the VH has at least 70%, 75%, 80%, 85%, 90%, 95%, 98%, 99% or 100% sequence identity to SEQ ID NO:7. In one embodiment, the VL has at least 70%, 75%, 80%, 85%, 90%, 95%, 98%, 99% or 100% sequence identity to SEQ ID NO:8.

In some embodiments, the 45H6E8 antibody is humanized. Example VH sequences of a humanized 45H6E8 antibody include SEQ ID NO:83-88. An example VL sequence is SEQ ID NO:89. In some embodiments, one or more of the CDRs of the 45H6E8 antibody is post-translation modification (PTM)-derisked. An example is SEQ ID NO:96 for the VH CDR2. Example VH sequences of a humanized, PTM-derisked 45H6E8 antibody include SEQ ID NO:91, 93 and 95.

In one embodiment, the VH has at least 70%, 75%, 80%, 85%, 90%, 95%, 98%, 99% or 100% sequence identity to SEQ ID NO:83. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:84. In one embodiment, the VL has at least 70%, 75%, 80%, 85%, 90%, 95%, 98%, 99% or 100% sequence identity to SEQ ID NO:89.

In one embodiment, the VH has at least 70%, 75%, 80%, 85%, 90%, 95%, 98%, 99% or 100% sequence identity to SEQ ID NO:85. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:86. In one embodiment, the VL has at least 70%, 75%, 80%, 85%, 90%, 95%, 98%, 99% or 100% sequence identity to SEQ ID NO:89.

In one embodiment, the VH has at least 70%, 75%, 80%, 85%, 90%, 95%, 98%, 99% or 100% sequence identity to SEQ ID NO:86. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:87. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:88. In one embodiment, the VL has at least 70%, 75%, 80%, 85%, 90%, 95%, 98%, 99% or 100% sequence identity to SEQ ID NO:89.

In one embodiment, the VH has at least 70%, 75%, 80%, 85%, 90%, 95%, 98%, 99% or 100% sequence identity to SEQ ID NO:91. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:93. In one embodiment, the VL has at least 70%, 75%, 80%, 85%, 90%, 95%, 98%, 99% or 100% sequence identity to SEQ ID NO:89. In one embodiment, the VH has at least 70%, 75%, 80%, 85%, 90%, 95%, 98%, 99% or 100% sequence identity to SEQ ID NO:95. In one embodiment, the VL has at least 70%, 75%, 80%, 85%, 90%, 95%, 98%, 99% or 100% sequence identity to SEQ ID NO:89.

Also provided, in some embodiments, are antibodies and antigen-binding fragments therefore that bind to the same epitope on CD16A as 45H6E8. Also provided, in some embodiments, are antibodies and antigen-binding fragments therefore that competes with 45H6E8 in binding to CD16A.

In one embodiment, an antibody or antigen binding fragment thereof of the present disclosure includes the CDR regions of antibody 23H7E4, which has a VH sequence of SEQ ID NO:1 and a VL sequence of SEQ ID NO:2.

In some embodiments, the VH CDR1, VH CDR2, VH CDR3, VL CDR1, VL CDR2, and VL CDR3 have the sequences of SEQ ID NO: 13, 14, 15, 28, 29 and 30, respectively. In some embodiments, the antibody or antigen binding fragment thereof of the present disclosure includes these CDR sequences and has at least 70%, 75%, 80%, 85%, 90%, 95%, 98%, 99% or 100% sequence identity to antibody 23H7E4. In one embodiment, the VH has at least 70%, 75%, 80%, 85%, 90%, 95%, 98%, 99% or 100% sequence identity to SEQ ID NO:1. In one embodiment, the VL has at least 70%, 75%, 80%, 85%, 90%, 95%, 98%, 99% or 100% sequence identity to SEQ ID NO:2.

Also provided, in some embodiments, are antibodies and antigen-binding fragments therefore that bind to the same epitope on CD16A as 23H7E4. Also provided, in some embodiments, are antibodies and antigen-binding fragments therefore that competes with 23H7E4 in binding to CD16A.

In one embodiment, an antibody or antigen binding fragment thereof of the present disclosure includes the CDR regions of antibody 37B3G11, which has a VH sequence of SEQ ID NO:3 and a VL sequence of SEQ ID NO:4.

In some embodiments, the VH CDR1, VH CDR2, VH CDR3, VL CDR1, VL CDR2, and VL CDR3 have the sequences of SEQ ID NO: 16, 17, 18, 31, 32 and 33, respectively. In some embodiments, the antibody or antigen binding fragment thereof of the present disclosure includes these CDR sequences and has at least 70%, 75%, 80%, 85%, 90%, 95%, 98%, 99% or 100% sequence identity to antibody 37B3G11. In one embodiment, the VH has at least 70%, 75%, 80%, 85%, 90%, 95%, 98%, 99% or 100% sequence identity to SEQ ID NO:3. In one embodiment, the VL has at least 70%, 75%, 80%, 85%, 90%, 95%, 98%, 99% or 100% sequence identity to SEQ ID NO:4.

Also provided, in some embodiments, are antibodies and antigen-binding fragments therefore that bind to the same epitope on CD16A as 37B3G11. Also provided, in some embodiments, are antibodies and antigen-binding fragments therefore that competes with 37B3G11 in binding to CD16A.

In one embodiment, an antibody or antigen binding fragment thereof of the present disclosure includes the CDR regions of antibody 42F5H2, which has a VH sequence of SEQ ID NO:5 and a VL sequence of SEQ ID NO:6.

In some embodiments, the VH CDR1, VH CDR2, VH CDR3, VL CDR1, VL CDR2, and VL CDR3 have the sequences of SEQ ID NO: 19, 20, 21, 34, 35 and 36, respectively. In some embodiments, the antibody or antigen binding fragment thereof of the present disclosure includes these CDR sequences and has at least 70%, 75%, 80%, 85%, 90%, 95%, 98%, 99% or 100% sequence identity to antibody 42F5H2. In one embodiment, the VH has at least 70%, 75%, 80%, 85%, 90%, 95%, 98%, 99% or 100% sequence identity to SEQ ID NO:5. In one embodiment, the VL has at least 70%, 75%, 80%, 85%, 90%, 95%, 98%, 99% or 100% sequence identity to SEQ ID NO:6.

Also provided, in some embodiments, are antibodies and antigen-binding fragments therefore that bind to the same epitope on CD16A as 42F5H2. Also provided, in some embodiments, are antibodies and antigen-binding fragments therefore that competes with 42F5H2 in binding to CD16A.

In one embodiment, an antibody or antigen binding fragment thereof of the present disclosure includes the CDR regions of antibody 91A11D11, which has a VH sequence of SEQ ID NO:9 and a VL sequence of SEQ ID NO:10.

In some embodiments, the VH CDR1, VH CDR2, VH CDR3, VL CDR1, VL CDR2, and VL CDR3 have the sequences of SEQ ID NO: 25, 26, 27, 40, 41 and 42, respectively. In some embodiments, the antibody or antigen binding fragment thereof of the present disclosure includes these CDR sequences and has at least 70%, 75%, 80%, 85%, 90%, 95%, 98%, 99% or 100% sequence identity to antibody 91A11D11. In one embodiment, the VH has at least 70%, 75%, 80%, 85%, 90%, 95%, 98%, 99% or 100% sequence identity to SEQ ID NO:9. In one embodiment, the VL has at least 70%, 75%, 80%, 85%, 90%, 95%, 98%, 99% or 100% sequence identity to SEQ ID NO:10.

Also provided, in some embodiments, are antibodies and antigen-binding fragments therefore that bind to the same epitope on CD16A as 91A11D11. Also provided, in some embodiments, are antibodies and antigen-binding fragments therefore that competes with 91A11D11 in binding to CD16A.

In some embodiments, the antibody of the present disclosure is a full-size IgG antibody, such as IgG1, IgG2, IgG3 or IgG4. In some embodiments, the antibody has an Fc fragment without effector function (e.g., not capable of binding to an Fcγ receptor or complement protein). Modifications to the wild-type Fc fragments are known that can deprive the Fc fragment such binding activities. An example is the IgG1 LALAPG mutant (L234A/L235A/P329G, EU numbering; SEQ ID NO:76).

Another example is IgG1 Fc with L234A/L235A (LALA) which substitutions reduce binding to the IgG Fc receptors FcγRI, FcγRII and FcγRIII as well as to complement component C1q. Yet additional examples are L234F/L235E/P331S (FES), L234F/L235Q/K322Q (FQQ), L234A/G237A, L234A/L235A/G237A, L234A/L235A/G237A/P238S/H268A/A330S/P330S, L234A/L235E, G236R/L328R, and L234A/L235A/K322A. For IgG2 Fc, an example is A330S/P331S (also EU numbering).

### Multi-Functional Molecules

Another embodiment of the present disclosure provides multi-specific/multi-functional antibodies that can activate CD16A selectively in a target-dependent manner. Such multi-functional molecules, with one portion binding to CD16A and another portion targeting a specific antigen on a target tumor cell, can exhibit efficient activation and durable ADCC/ADCP function of NK cells and macrophages.

Accordingly, one embodiment provides a multi-specific antibody that includes an anti-CD16A antibody or antigen-binding fragment having binding specificity to the human CD16A protein; and a second antibody or antigen-binding fragment having binding specificity to a tumor-associated antigen (TAA), which can also be referred to as an anti-TAA antibody or antigen binding fragment.

In some embodiments, the multi-specific antibody includes an Fc fragment, which can be an Fc fragment of human IgG1, IgG2, IgG3 or IgG4, or modified isoforms thereof.

In one embodiment, the Fc fragment has reduced or no effector function, which can be achieved with Fc mutation(s) that reduce or eliminate binding to Fcγ receptor or complement proteins.

Example mutations that reduce or eliminate Fc's effector functions include, without limitation, IgG1 L234A/L235A (LALA), L234A/L235A/P329G (LALAPG), L234F/L235E/P331S (FES), L234F/L235Q/K322Q (FQQ), L234A/G237A, L234A/L235A/G237A, 234A/L235A/G237A/P238S/H268A/A330S/P330S, L234A/L235E, G236R/L328R, and L234A/L235A/K322A, and IgG2 A330S/P331S (EU numbering). In a preferred embodiment, the Fc fragment is IgG1 LALAPG (SEQ ID NO:76).

In another embodiment, the Fc fragment is a wild-type Fc, such as wildtype IgG1 Fc, wildtype IgG2 Fc, wildtype IgG3 Fc, or wildtype IgG4 Fc. In a particular embodiment, the Fc is a wildtype IgG1 Fc. In certain embodiments, the Fc fragment does not include mutations that can enhance the effector function of the antibody. In some embodiments, the Fc fragment does not include post-translational modifications that can enhance the effector function of the antibody.

As demonstrated in the experimental examples, the natural effector functions of these wildtype Fc fragment can synergize with the anti-CD16A antibody/fragment. Such Fc fragments can bind to CD64 and does not bind CD16B or CD32B which can inhibit or reduce ADCC activities. Enhanced effector functions, such as with mutations such as Fc-DLE (S239D/A330L/I332E) or Fc-DE (S239D/I332E), can cause or increase the binding to CD16B and/or CD32B, leading to reduced ADCC effects.

The multi-specific antibody can take any format, in which the anti-CD16A and/or anti-TAA portions may include one, two, three or four antigen binding fragment(s). In some embodiments, the anti-CD16A portion and the anti-TAA portion may be at the same side (e.g., N-terminal) of the Fc fragment.

In some embodiments, the anti-CD16A portion and the anti-TAA portion may be at the opposite sides of the Fc fragment. In one embodiment, the anti-CD16A portion is at the N-terminal side of the Fc, and the anti-TAA portion is at the C-terminal side of the Fc. In another embodiment, the anti-CD16A portion is at the C-terminal side of the Fc, and the anti-TAA portion is at the N-terminal side of the Fc.

When the anti-TAA portion is at the N-terminus of the Fc fragment, it can form a conventional full-sized IgG antibody with the Fc fragment, while the anti-CD16A portion, which may be in the form of two separate single chain fragments (scFv), is fused to the C-terminus of the Fc fragment.

The anti-CD16A antibody or fragment may be any known anti-CD16A antibody or fragment such as P2C47, or the currently identified ones, including 45H6E8, 23H7E4, 37B3G11, 42F5H2, and 91A11D11.

In some embodiments, the anti-CD16A antibody or fragment preferably has minimum or no affinity to the human CD16B protein, such as 45H6E8.

Example anti-CD16A antibodies or fragments include CDR sequences of antibodies shown in Tables 1, 6A and 7A. Representative CDR sequences (Kabat numbering) of these antibodies are shown in Tables 1A-B and 7B, such as those in SEQ ID NO: 22, 96, 24, 37, 38 and 39; SEQ ID NO: 22, 23, 24, 37, 38 and 39; SEQ ID NO: 13, 14, 15, 28, 29 and 30; SEQ ID NO: 16, 17, 18, 31, 32, and 33; SEQ ID NO: 19, 20, 21, 34, 35 and 36; or SEQ ID NO: 25, 26, 27, 40, 41, and 42. Their biological variants, including optimized and humanized counterparts, are further described in the sections above and are incorporated herein.

In some embodiments, the TAA is selected from the group consisting of GPC3, Claudin 18.2, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin. In some embodiments, the TAA is GPC3.

An example anti-GPC3 antibody has been prepared and tested in the experimental example, which includes a VH of SEQ ID NO:56 and a VL of SEQ ID NO:57. It is to be appreciated that other anti-GPC3 antibodies and variants can also be used herein.

Example multi-specific antibodies have been prepared and tested herein, such as those with sequences provided in Table 9A. In some embodiments, the multi-specific antibody includes a heavy chain having the amino acid sequence of SEQ ID NO:71 and a light chain having the amino acid sequence of SEQ ID NO:61. In some embodiments, the multi-specific antibody includes a heavy chain having the amino acid sequence of SEQ ID NO:65 and a light chain having the amino acid sequence of SEQ ID NO:61. In some embodiments, the multi-specific antibody includes a heavy chain having the amino acid sequence of SEQ ID NO:67 and a light chain having the amino acid sequence of SEQ ID NO:61. In some embodiments, the multi-specific antibody includes a heavy chain having the amino acid sequence of SEQ ID NO:69 and a light chain having the amino acid sequence of SEQ ID NO:61. In some embodiments, the multi-specific antibody includes a heavy chain having the amino acid sequence of SEQ ID NO:73 and a light chain having the amino acid sequence of SEQ ID NO:61.

In some embodiments, the multi-specific antibody includes a heavy chain having the amino acid sequence of SEQ ID NO:75 and a light chain having the amino acid sequence of SEQ ID NO:61.

Example multi-specific antibodies have been prepared and tested herein, such as those with sequences provided in Table 9B. In some embodiments, the multi-specific antibody includes a heavy chain having the amino acid sequence of SEQ ID NO:97 and a light chain having the amino acid sequence of SEQ ID NO:98. In some embodiments, the multi-specific antibody includes a heavy chain having the amino acid sequence of SEQ ID NO:100 and a light chain having the amino acid sequence of SEQ ID NO:101. In some embodiments, the multi-specific antibody includes a heavy chain having the amino acid sequence of SEQ ID NO:103 and a light chain having the amino acid sequence of SEQ ID NO:104. In some embodiments, the multi-specific antibody includes a heavy chain having the amino acid sequence of SEQ ID NO:106 and a light chain having the amino acid sequence of SEQ ID NO:107. In some embodiments, the multi-specific antibody includes a heavy chain having the amino acid sequence of SEQ ID NO:109 and a light chain having the amino acid sequence of SEQ ID NO:110.

Example multi-specific antibodies have been prepared and tested herein, such as those with sequences provided in Table 9C. In some embodiments, the multi-specific antibody includes a heavy chain having the amino acid sequence of SEQ ID NO:112 and a light chain having the amino acid sequence of SEQ ID NO:113. In some embodiments, the multi-specific antibody includes a heavy chain having the amino acid sequence of SEQ ID NO:115 and a light chain having the amino acid sequence of SEQ ID NO:116.

An example anti-CCR8 antibody has been prepared and tested in the experimental example, which includes a VH of SEQ ID NO:117 and a VL of SEQ ID NO:118. It is to be appreciated that other anti-CCR8 antibodies and variants can also be used herein.

Example multi-specific antibodies have been prepared and tested herein, such as those with sequences provided in Tables 10B. In some embodiments, the multi-specific antibody includes a heavy chain having the amino acid sequence of SEQ ID NO:119 and a light chain having the amino acid sequence of SEQ ID NO:120. In some embodiments, the multi-specific antibody includes a heavy chain having the amino acid sequence of SEQ ID NO:121 and a light chain having the amino acid sequence of SEQ ID NO:122. In some embodiments, the multi-specific antibody includes a heavy chain having the amino acid sequence of SEQ ID NO:123 and a light chain having the amino acid sequence of SEQ ID NO:124. In some embodiments, the multi-specific antibody includes a heavy chain having the amino acid sequence of SEQ ID NO:125 and a light chain having the amino acid sequence of SEQ ID NO:126. In some embodiments, the multi-specific antibody includes a heavy chain having the amino acid sequence of SEQ ID NO:127 and a light chain having the amino acid sequence of SEQ ID NO:128. In some embodiments, the multi-specific antibody includes a heavy chain having the amino acid sequence of SEQ ID NO:129 and a light chain having the amino acid sequence of SEQ ID NO:130. In some embodiments, the multi-specific antibody includes a heavy chain having the amino acid sequence of SEQ ID NO:131 and a light chain having the amino acid sequence of SEQ ID NO:132. In some embodiments, the multi-specific antibody includes a heavy chain having the amino acid sequence of SEQ ID NO:133 and a light chain having the amino acid sequence of SEQ ID NO:134. In some embodiments, the multi-specific antibody includes a heavy chain having the amino acid sequence of SEQ ID NO:135 and a light chain having the amino acid sequence of SEQ ID NO:136.

An example anti-CCR8 antibody has been prepared and tested in the experimental example, which includes a VH of SEQ ID NO:77 and a VL of SEQ ID NO:78. It is to be appreciated that other anti-CCR8 antibodies and variants can also be used herein.

Example multi-specific antibodies have been prepared and tested herein, such as those with sequences provided in Table 12. In some embodiments, the multi-specific antibody includes a heavy chain having the amino acid sequence of SEQ ID NO:79 and a light chain having the amino acid sequence of SEQ ID NO:80. In some embodiments, the multi-specific antibody includes a heavy chain having the amino acid sequence of SEQ ID NO:81 and a light chain having the amino acid sequence of SEQ ID NO:80. In some embodiments, the multi-specific antibody includes a heavy chain having the amino acid sequence of SEQ ID NO:82 and a light chain having the amino acid sequence of SEQ ID NO:80.

In certain embodiments, the antibodies (including multi-specific or multi-functional antibody) or fragments thereof includes an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

The antibodies (including multi-specific or multi-functional antibody) or fragments thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Treatment and Uses

As described herein, the antibodies (including multi-specific or multi-functional antibody) or fragments thereof of the disclosure may be used in certain treatment and diagnostic methods for diseases or conditions such as cancer.

Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient expresses, over-express, or is induced to express a tumor antigen that the multi-specific antibody recognizes (e.g., a TAA such as GPC3). Induction of a gene expression, for instance, can be done by administration of a tumor vaccine or radiotherapy.

Tumors that can be suitably treated include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers.

In some embodiments, the tumors being treated are those that are particularly challenging to treat with conventional immuno-oncological therapies, such as with antibodies targeting immune checkpoints (ICPs). Sometimes, such tumors are referred to as “cold tumors” or “nonimmunogenic tumors.” In some embodiments, accordingly, the present disclosure provides methods and uses for treating cold tumors with multi-specific antibodies disclosed herein.

In some embodiments, a nonimmunogenic tumor is one that is not infiltrated with T cells, or that is deficient in T cell infiltration, in antigen presenting cells (APCs), or in T cell activation, or has deficit in T cell homing into the tumor bed. All of prostate cancer, pancreatic cancer, and leukemia are nonimmunogenic. The vast majority of breast cancer (95%), colorectal cancer (95%), gastric cancer (87%), head and neck cancer (84%), liver cancer (83%), esophageal cancer (86%), cervical cancer (87%), and thyroid cancer (87%) are also nonimmunogenic. In addition, 83% of lung cancer, 79% of bladder cancer, 77% of kidney cancer, 70% uterus cancer, and 66% melanoma are also nonimmunogenic.

Identification of nonimmunogenic, or cold tumors can also be made with measurements of type, density and location of immune cells within the tumors. For instance, Galon and Bruni (Nature Reviews Drug Discovery volume 18, pages 197-218 (2019)) describes a standardized scoring system, Immunoscore, based on the quantification of two lymphocyte populations (CD3 and CD8), e.g., in resected tissues, for guided stratification of hot and cold tumors. The Immunoscore ranges from Immunoscore 0 (IO, for low densities, such as absence of both cell types in both regions) to 14 (high immune cell densities in both locations). By classifying cancers according to their immune infiltration, the scoring system provides an immune-based classification of tumors, including a definition of “hot” (highly infiltrated, Immunoscore 14) and “cold” (non-infiltrated, Immunoscore IO) tumors.

In some embodiments, the tumor is resistant to a treatment with immune checkpoint inhibitors, such as PD-L1 inhibitors, PD-1 inhibitors, CTLA-4 inhibitors, or the combinations thereof. In some embodiments, the cancer is prostate cancer, pancreatic cancer, or leukemia. In some embodiments, the cancer is breast cancer, colorectal cancer, gastric cancer, head and neck cancer, liver cancer, esophageal cancer, cervical cancer, or thyroid cancer. In some embodiments, the cancer is lung cancer, bladder cancer, kidney cancer, uterus cancer, or melanoma.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antibodies polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

In certain embodiments, the prepared antibodies will not elicit a deleterious immune response in the animal to be treated, e.g., in a human. In one embodiment, antigen-binding polypeptides, variants, or derivatives thereof of the disclosure are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues.

De-immunization can also be used to decrease the immunogenicity of an antibody. As used herein, the term “de-immunization” includes alteration of an antibody to modify T-cell epitopes (see, e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of antigen-binding polypeptides of the present disclosure can be determined by in vitro assays such as immunoprecipitation, radioimmunoassay (RIA) or enzyme-linked immunoabsorbent assay (ELISA).

### Compositions

The present disclosure also provides pharmaceutical compositions. Such compositions comprise an effective amount of an antibody (including a multi-specific antibody), and an acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1. Generation of Mouse Anti Human CD16A Antibody

This example describes the generation of mouse anti-human CD16A monoclonal antibodies using the hybridoma technology.

Immuogen: Two immunogens were used during the mouse immunization process. An extracellular domain (ECD) of human CD16A-158V was either fused with human IgG1 Fc region with N297A mutation or his tag at the C-terminus to generate hCD16A-158V-FcNA protein (Biointron) or hCD16A-158V-his protein (AcroBio, Cat: CD8-H52H4).

Scheme of mouse immunization: To generate mouse monoclonal antibodies to human CD16A, BALB/c mice were immunized alternatively with hCD16A-158V-FcNA or hCD16A-158V-his protein at a biweekly interval intraperitoneally and subcutaneously. Serum titers of immunized mice were monitored by ELISA against human CD16A-158V-his protein. After 2-4 rounds of immunization, mice with sufficient titers were boosted with hCD16A-158V-his protein and selected for fusions.

Cellfusion and Hybridoma screening: Splenocytes from the selected mice were fused with mouse myeloma cell line Sp2/0 by electrofusion. These hybridoma cells were plated in 96 flat-bottom microplates and secreted mouse antibodies in the supernatant. During primary screening, protein-based binding to hCD16A-158V-his by ELISA or cell-based binding to hCD16A-158V overexpressed on CHO-K1 cells (CHO-K1-hCD16A-158V) by Mirrorball (SPT Labtech) was used to screen positive clones in a high-throughput way. A following confirmative screening was pursued to rule out the clones with non-specific binding to hCD16B-SH-his protein (SinoBiological, Cat: NO. 11046-H08H2) by ELISA or hCD16B-NA1 over-expressed on CHO-K1 cells (CHO-K1-hCD16B-NA1) by FACS and identify the clones binding to cynomolgus CD16 over-expressed on CHO-K1 cells (CHO-K1-cynoCD16).

Subcloning screening and sequencing: Positive primary clones from each fusion were subcloned by limiting dilutions to ensure that hybridoma subclones were derived from a single parental cell. Subcloning were screened with the same criteria as primary clone screening as described above. The subclones with specific binding potency to hCD16A and no binding to hCD16B were selected for subsequent sequencing.

The resulting sequences of the Fab fragment of the mouse antibody were fused with human IgG1 Fc with L234A/L235A/P329G (LALAPG) mutations to generate chimeric CD16A mAbs. In order to validate the CD16A agonistic efficacy exerted by Fab fragment of the CD16A mAbs, LALAPG mutations were introduced in the Fc region which completely abolished the Fc binding to all the FcγRs and the resulting ADCC and ADCP effect. The DNA sequences of chimeric antibodies were cloned into the pcDNA3.4 plasmid and expressed in HEK-293F or CHO-K1 cells followed by purification of antibodies from the culture supernatant by Protein A affinity chromatography column or beads. The purified chimeric antibodies were subjected to serial in vitro screening processes to determine affinity, binding ability, specificity, species cross-reactivity and agonistic function.

Several hybridoma clones including 23H7E4, 37B3G11, 42F5H2, 45H6E8 and 91A11D11 were selected for further analysis based on the performance in the screening assays. The amino acid sequences of the variable regions of the selected mouse CD16A antibodies are provided in Table 1 as below, with the CDR sequences summarized in Table 1A and 1B. An affinity matured CD16A mAb P2C47 (sequence derived from U.S. Ser. No. 11/001,633B2) which has been developed by Affimed was used as the benchmark antibody with the sequence of the Fab fragment listed in Table 1. The sequences of the heavy chain and light chain of the antibodies are listed in Table 2.

TABLE 1
Antibody Variable Region Sequences (Underline indicates CDRs)

| | | SEQ |
|---|---|---|
| Name | Sequence | ID NO: |
| 23H7E4 VH | QVQLQQPGAELVRPGASVKLSCKASGYTFTSYWLNWVKQRPGQGLEWIGM | 1 |
| | IDPSDSETHYNQVFKDKATLTVDKSSTTAYMQLSSLTSEDSAVYYCARWM | |
| | VTKKTFDYWGQGTTLTVSS | |
| 23H7E4 VL | DIVLTQSPASLAVSLGQRATISCRASESVEYYGPSLMQWYQQKPGQPPKL | 2 |
| | LIYAASNVESGVPARFSGSGSGTDFSLNIHPVEEDDIAMYFCQQSRKVPY | |
| | TFGGGAKLEIK | |
| 37B3G11 VH | QVQLQQPGAELVRPGASVKLSCKASGYTFTTYWMNWVKQRPGQGLEWIGM | 3 |
| | IDPSDSETHYNQMFKDKATLTVDKSSSTAYMQLSSLTSEDSAVYYCAVPS | |
| | LYNSNYRFAYWGQGTLVTVSA | |
| 37B3G11 VL | DIVLTQSPASLAVSLGQRATISCRASESVEYYGTSLMQWYQQKPGQPPKL | 4 |
| | LIYSASNVESGVPARFSGSGSGTDFSLNIHPVEEDDIAMYFCQQSRKVPW | |
| | TFGGGTKLEIK | |
| 42F5H2 VH | QVQLQQPGAELVRPGASVKLSCKASGYTFTSYWMNWVKQRPGQGLEWIGM | 5 |
| | IDPSDSETHHNQMFKDKATLTVDKSSSTAYMQLSSLTSEDSAVYYCAKWL | |
| | PPYAMDYWGQGTSVTVSS | |
| 42F5H2 VL | DIVLTQSPASLAVSLGQRATISCRASESVEYYGPSLMQWYQQKPGQPPKL | 6 |
| | LIYAASNVESGVPARFSGSGSGTDFSLNIHPVEEDDIAMYFCQQSRKVPY | |
| | TFGGGTKLEIK | |
| 45H6E8 VH | EVQLQQSGAELVRSGASVKLSCTASGFNIKDYYMQWVKQRPEQGLEWIGW | 7 |
| | IDPENGDTEYAPKFQGKATMTADTSSNTAYLQLSSLTSEDTAVYYCTTGG | |
| | NYVDWGQGTTLTVSS | |
| 45H6E8 VL | DIVMTQSQKFMSTSVGDRVSITCKASQNVGTAVAWYQQKPGQSPKLLIYS | 8 |
| | ASNRYTGVPDRFTGSGSGTDFTLTISNMQSEDLADYFCQQYSSYPLLTFG | |
| | AGTNLELK | |
| 91A11D11 | QVQLQQPGAELVRPGTSVKLSCKASGYTFTSYWMNWVKQRPGQGLEWIGM | 9 |
| VH | IDPSDSETHYNQMFKDKATLTVDESSSTAYMQLSSLTSEDSAVYFCARWT | |
| | LVVAHFFDYWGQGTTLTVSS | |
| 91A11D11 | DIVLTQSPASLAVSLGQRATISCRASETVEYYGPSLLQWYQQKPGQPPKL | 10 |
| VL | LIYGASNVESGVPARFSGSGSGTDFSLNIHPVEEDDVAMYFCQQSRKVPY | |
| | TFGGGTKLEIK | |
| P2C47 VH | QVQLVQSGAEVKKPGESLKVSCKASGYTFTSYYMHWVRQAPGQGLEWMGA | 11 |
| | IEPMYGSTSYAQKFQGRVTMTRDTSTSTVYMELSSLRSEDTAVYYCARGS | |
| | AYYYDFADYWGQGTLVTVSS | |
| P2C47 VL | SYVLTQPSSVSVAPGQTATISCGGHNIGSKNVHWYQQRPGQSPVLVIYQD | 12 |
| | NKRPSGIPERFSGSNSGNTATLTISGTQAMDEADYYCQVWDNYSVLFGGG | |
| | TKLTVL | |

TABLE 1A
Heavy Chain CDR Sequences (Kabat numbering)

| | | SEQ | | SEQ | | SEQ |
|---|---|---|---|---|---|---|
| | VH | ID | | ID | | ID |
| Antibody | CDR1 | NO: | VH CDR2 | NO: | VH CDR3 | NO: |
| 23H7E4 | SYWLN | 13 | MIDPSDSETHYNQVFKD | 14 | WMVTKKTFDY | 15 |
| 37B3G11 | TYWMN | 16 | MIDPSDSETHYNQMFKD | 17 | PSLYNSNYRFAY | 18 |
| 42F5H2 | SYWMN | 19 | MIDPSDSETHHNQMFKD | 20 | WLPPYAMDY | 21 |
| 45H6E8 | DYYMQ | 22 | WIDPENGDTEYAPKFQG | 23 | GGNYVD | 24 |
| 91A11D11 | SYWMN | 25 | MIDPSDSETHYNQMFKD | 26 | WTLVVAHFFDY | 27 |

TABLE 1B
Light Chain CDR Sequences (Kabat numbering)

| | | SEQ | | SEQ | | SEQ |
|---|---|---|---|---|---|---|
| | | ID | | ID | | ID |
| Antibody | VL CDR1 | NO: | VL CDR2 | NO: | VL CDR3 | NO: |
| 23H7E4 | RASESVEYYGPSLMQ | 28 | AASNVES | 29 | QQSRKVPYT | 30 |
| 37B3G11 | RASESVEYYGTSLMQ | 31 | SASNVES | 32 | QQSRKVPWT | 33 |
| 42F5H2 | RASESVEYYGPSLMQ | 34 | AASNVES | 35 | QQSRKVPYT | 36 |
| 45H6E8 | KASQNVGTAVA | 37 | SASNRYT | 38 | QQYSSYPLLT | 39 |
| 91A11D11 | RASETVEYYGPSLLQ | 40 | GASNVES | 41 | QQSRKVPYT | 42 |

TABLE 2
CD16A Chimeric Ab Sequences

| | | SEQ |
|---|---|---|
| Name | Sequence | ID NO: |
| 23H7E4-hIgG1 | QVQLQQPGAELVRPGASVKLSCKASGYTFTSYWLNWVKQRPGQGLEWIGMI | 43 |
| LALAPG | DPSDSETHYNQVFKDKATLTVDKSSTTAYMQLSSLTSEDSAVYYCARWMVT | |
| Heavy Chain | KKTFDYWGQGTTLTVSS | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| 23H7E4-hIgG1 | DIVLTQSPASLAVSLGQRATISCRASESVEYYGPSLMQWYQQKPGQPPKLL | 44 |
| LALAPG Light | IYAASNVESGVPARFSGSGSGTDFSLNIHPVEEDDIAMYFCQQSRKVPYTF | |
| Chain | GGGAKLEIK | |
| | RTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSF | |
| | NRGEC | |
| 37B3G11- | QVQLQQPGAELVRPGASVKLSCKASGYTFTTYWMNWVKQRPGQGLEWIGMI | 45 |
| hIgG1 LALAPG | DPSDSETHYNQMFKDKATLTVDKSSSTAYMQLSSLTSEDSAVYYCAVPSLY | |
| Heavy Chain | NSNYRFAYWGQGTLVTVSA | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| 37B3G11- | DIVLTQSPASLAVSLGQRATISCRASESVEYYGTSLMQWYQQKPGQPPKLL | 46 |
| hIgG1 LALAPG | IYSASNVESGVPARFSGSGSGTDFSLNIHPVEEDDIAMYFCQQSRKVPWTF | |
| Light Chain | GGGTKLEIK | |
| | RTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSF | |
| | NRGEC | |
| 42F5H2-hIgG1 | QVQLQQPGAELVRPGASVKLSCKASGYTFTSYWMNWVKQRPGQGLEWIGMI | 47 |
| LALAPG | DPSDSETHHNQMFKDKATLTVDKSSSTAYMQLSSLTSEDSAVYYCAKWLPP | |
| Heavy Chain | YAMDYWGQGTSVTVSS | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| 42F5H2-hIgG1 | DIVLTQSPASLAVSLGQRATISCRASESVEYYGPSLMQWYQQKPGQPPKLL | 48 |
| LALAPG Light | IYAASNVESGVPARFSGSGSGTDFSLNIHPVEEDDIAMYFCQQSRKVPYTF | |
| Chain | GGGTKLEIK | |
| | RTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSF | |
| | NRGEC | |
| 45H6E8-hIgG1 | EVQLQQSGAELVRSGASVKLSCTASGFNIKDYYMQWVKQRPEQGLEWIGWI | 49 |
| LALAPG | DPENGDTEYAPKFQGKATMTADTSSNTAYLQLSSLTSEDTAVYYCTTGGNY | |
| Heavy Chain | VDWGQGTTLTVSS | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| 45H6E8-hIgG1 | DIVMTQSQKFMSTSVGDRVSITCKASQNVGTAVAWYQQKPGQSPKLLIYSA | 50 |
| LALAPG Light | SNRYTGVPDRFTGSGSGTDFTLTISNMQSEDLADYFCQQYSSYPLLTFGAG | |
| Chain | TNLELK | |
| | RTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSF | |
| | NRGEC | |
| 91A11D11- | QVQLQQPGAELVRPGTSVKLSCKASGYTFTSYWMNWVKQRPGQGLEWIGMI | 51 |
| hIgG1 LALAPG | DPSDSETHYNQMFKDKATLTVDESSSTAYMQLSSLTSEDSAVYFCARWTLV | |
| Heavy Chain | VAHFFDYWGQGTTLTVSS | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| 91A11D11- | DIVLTQSPASLAVSLGQRATISCRASETVEYYGPSLLQWYQQKPGQPPKLL | 52 |
| hIgG1 LALAPG | IYGASNVESGVPARFSGSGSGTDFSLNIHPVEEDDVAMYFCQQSRKVPYTF | |
| Light Chain | GGGTKLEIK | |
| | RTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSF | |
| | NRGEC | |
| P2C47-hIgG1 | QVQLVQSGAEVKKPGESLKVSCKASGYTFTSYYMHWVRQAPGQGLEWMGAI | 53 |
| LALAPG | EPMYGSTSYAQKFQGRVTMTRDTSTSTVYMELSSLRSEDTAVYYCARGSAY | |
| Heavy Chain | YYDFADYWGQGTLVTVSS | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKS | |
| | CDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALGAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| P2C47-hIgG1 | SYVLTQPSSVSVAPGQTATISCGGHNIGSKNVHWYQQRPGQSPVLVIYQDN | 54 |
| LALAPG | KRPSGIPERFSGSNSGNTATLTISGTQAMDEADYYCQVWDNYSVLFGGGTK | |
| Light Chain | LTVL | |
| | GQPKANPTVTLFPPSSEELQANKATLVCLISDFYPGAVTVAWKADGSPVKA | |
| | GVETTKPSKQSNNKYAASSYLSLTPEQWKSHRSYSCQVTHEGSTVEKTVAP | |
| | TECS | |

### Example 2. Binding Activities of Chimeric Anti-Human CD16A Monoclonal Antibodies

### 2.1. ELISA Binding to Human CD16A

In order to determine the binding ability of the chimeric Abs to human CD16A protein, ELISA-based binding assay was performed as followed. In brief, hCD16A-158V-his and hCD16A-158F-his protein (Acro Bio, Cat: NO. CDA-H5220) were diluted with DPBS buffer at 2 μg/mL and adsorbed to wells of 96-well microplates overnight at 4° C. After blocking the wells with 2% bovine serum albumin (BSA) to prevent non-specific binding, CD16A chimeric Abs, benchmark antibody P2C47, or an isotype control were titrated at a 3-fold dilution rate starting from 20 nM and added to the wells pre-adsorbed with hCD16A-his protein. The mixture was incubated for 1 hour at room temperature (RT). The primary antibody was recognized by a detective antibody against human IgG Fc which was conjugated with horseradish peroxidase (HRP) (Jackson Immuno, Cat. No. 109-035-008). Tetramethylbenzidine (TMB), a substrate of HRP, was added to the wells to visualize the binding signal. After sufficient color development, stop solution was added to the wells. The absorbance of the signal was detected at 450 nm with Envision multilabel plate readers (PerkinElemer). The graph and statistics analysis were generated with four-parameter nonlinear regression curve fit in Graphpad Prism 9 software.

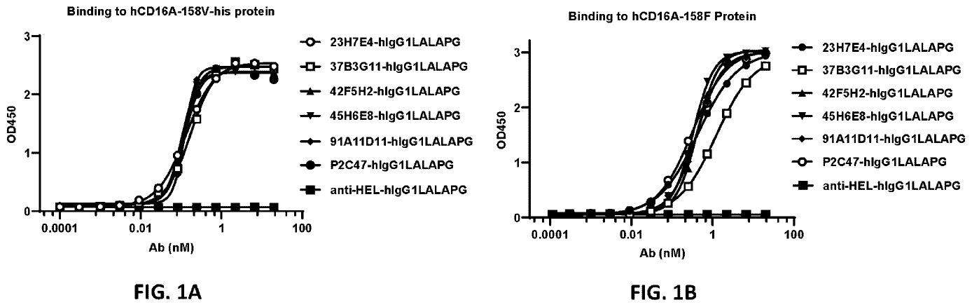

As shown in FIGS. 1A and 1B, all the CD16A chimeric Abs efficiently bound to the high-affinity 158V and low-affinity 158F of human CD16A protein. Furthermore, all the antibodies exhibit comparable binding efficiency to that of the benchmark antibody P2C47 except for 37B3G11 chimeric Ab. The binding EC₅₀ of tested CD16A chimeric Abs are listed in Table 3.

### 2.2. ELISA Binding to Human CD16B

It is crucial to identify the specificity of CD16A chimeric antibodies due to that only very few amino acids could differentiate CD16A with its isoform CD16B which makes it extremely challenging to generate a CD16A-specific antibody.

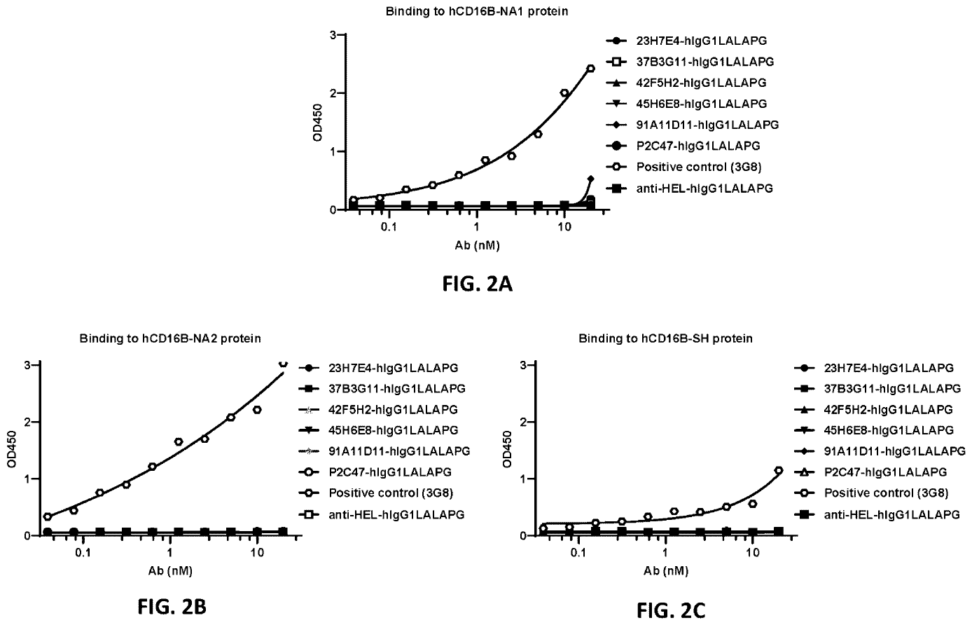

In order to determine the binding ability of the chimeric Abs to human CD16B protein, ELISA binding assay was performed as previously described. Three spliced isoforms of CD16B including human CD16B-NA1 ECD his tag protein (hCD16B-NA1-his, Acro Bio, Cat: No. CDB-H5227), human CD16B-NA2 ECD his tag protein (hCD16B-NA2-his, Acro Bio, Cat: No. CDB-H82Ea) and human CD16B-SH-his protein, were used as the coating antigens at 2 μg/mL. A commercial anti-CD16 antibody 3G8 (Stemcell, Cat No. 60041) with recognition to hCD16B was used as positive control. As shown in FIGS. 2A, 2B and 2C, all the CD16A chimeric Abs displayed neglectable binding to hCD16B-NA1, hCD16B-NA2 protein or hCD16B-SH protein, similar to the benchmark antibody, indicating the strict binding specificity of these chimeric CD16A antibodies recognizing a distinguished epitope on CD16A but not CD16B.

### 2.3 ELISA Binding to Cynomolgus CD16 Protein

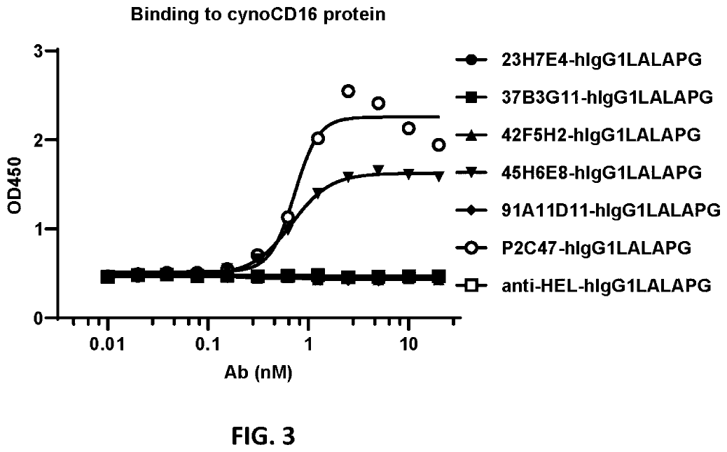

In order to determine the cross reactivity of the chimeric Abs to cynomolgus CD16, ELISA binding assay was performed as described above. Recombinant cyno CD16 ECD his tag protein (cynoCD16-his, Acro Bio, Cat. No. FC6-C52H9) was used as the coating antigen at 2 μg/mL. As shown in FIG. 3, only 45H6E8 bound efficiently to cyno CD16 protein, while other chimeric Abs including 23H7E4, 37B3G11,42F5H2 and 91A11D11 exhibited marginal binding to cyno CD16 protein. These data were probably due to that only extremely limited amino acids could distinguish human CD16A and cyno CD16 from all three isoforms of human CD16B. Nevertheless, 45H6E8 is a specific CD16A antibody that showed excellent cross-reactivity between human and cynomolgus species as well as precise specificity to two human CD16A polymorphisms.

TABLE 3
Binding activities of the chimeric antibodies to antigen protein

| | Antigen | | | |
|---|---|---|---|---|
| | | | Human | |
| | Human | Human | CD16B- | Cynomolgus |
| | CD16A-158V | CD16A-158F | NA1/NA2/SH | CD16 |
| Antibody | EC50 (nM) | | | |
| 23H7E4 | 0.13 | 0.44 | — | — |
| 37B3G11 | 0.17 | 1.20 | — | — |
| 42F5H2 | 0.11 | 0.42 | — | — |
| 45H6E8 | 0.10 | 0.33 | — | 0.68 |
| 91A11D11 | 0.12 | 0.41 | — | — |
| P2C47 | 0.12 | 0.32 | — | 0.73 |

—: No binding

### 2.4 Binding Ability to Human CD16A Expressed on CHO-K1 Cells

In order to evaluate the binding activity of the chimeric mAbs to human CD16A expressed on cells, cell-based binding assay was employed as followed. Briefly, CHO-K1 cells stably expressing hCD16A-158V or hCD16A-158F variants were constructed. The indicated CD16A chimeric Abs, the benchmark antibody P2C47, or an isotype control were diluted at a four-fold dilution rate starting from a concentration of 100 nM in the staining buffer (DPBS buffer containing 2% BSA). The antibody dilutions were incubated with 1×10⁵ indicated cells in the 96-well microplate for one hour at 4° C. The antibodies binding to the antigen on the cell surface were detected with PE fluorophore-conjugated anti-human IgG Fc secondary antibodies (ThermoFisher scientific, Cat. No. 12-4998-82) at a dilution rate of 1:1000. Cells were analyzed by flow cytometer LSRFortessa Cell Analyzer (BD Biosciences). Data were analyzed with Flowjo 10.0 software. The graph and statistics analysis were generated with four-parameter nonlinear regression curve fit in Graphpad Prism 9 software.

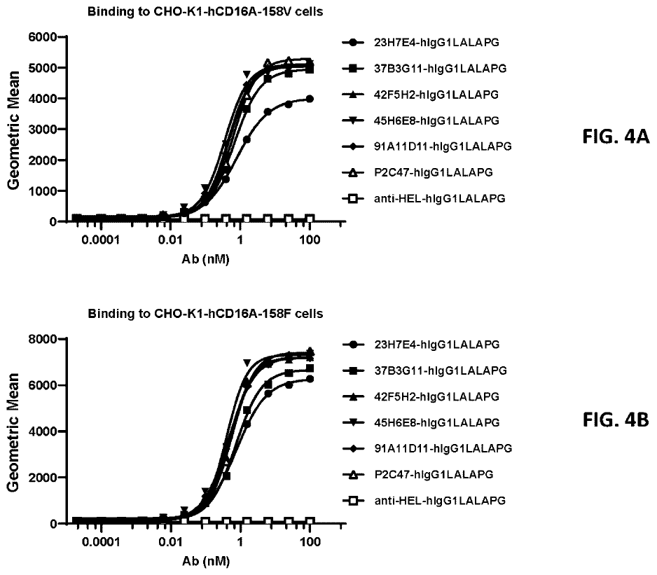

As shown in FIGS. 4A and 4B, all the CD16A chimeric antibodies efficiently bound to human CD16A-158V and CD16A-158F expressed on CHO-K1 cells in a dose-dependent manner. Most of our CD16A chimeric antibodies except for 23H7E4 exhibited comparable binding potency ability when compared with the benchmark antibody P2C47. The binding EC₅₀ of tested CD16A chimeric Abs are listed in Table 4.

### 2.5. Binding Ability to Human CD16B and CD32B Expressed on CHO-K1 Cells

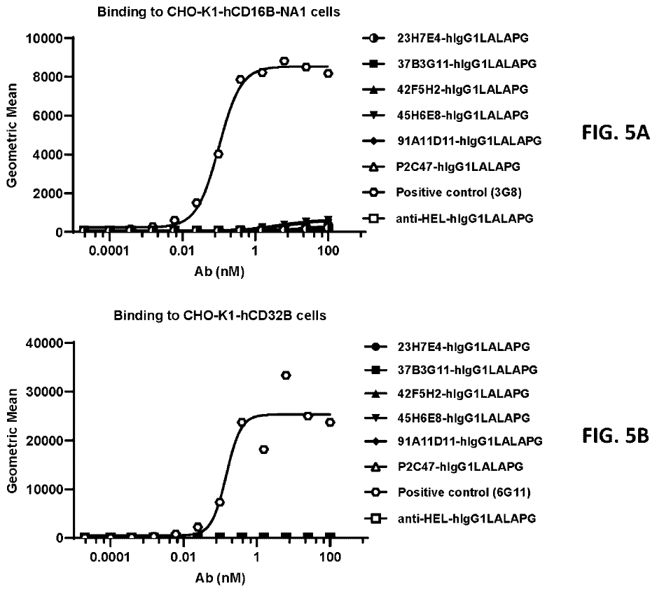

To exclude the non-specific binding of the CD16A chimeric antibody to human CD16B and CD32B expressed on cells, cell-based binding assay was performed as described above. Firstly, CHO-K1 cells were stably transferred with one human CD16B spliced isoform hCD16B-NA1 and human CD32B. Then, the cells were incubated with the indicated concentrations of CD16A chimeric antibodies. The commercial anti-CD16 antibody 3G8 with recognition to hCD16B and anti-CD32B antibody 6G11(Bioinvent) were used as the positive control. As shown in FIGS. 5A and 5B, all the CD16A chimeric antibodies showed minimal binding to hCD16B-NA1 and hCD32B expressed on the cells. Taken together, we have confirmed that our CD16A chimeric antibodies display a stringent binding specificity to human CD16A while avoiding non-specific binding of all the three isoforms of human CD16B and CD32B (FIG. 2 and FIG. 5).

### 2.6. Binding Ability to Cynomolgus CD16 on CHO-K1 Cells

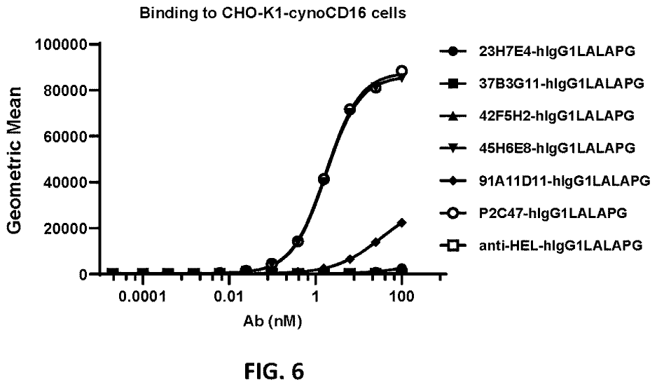

In order to confirm the cross reactivity of the chimeric CD16A antibodies to cynomolgus CD16, cell binding assay was performed as described above. CHO-K1 cells stably expressing cynomolgus CD16 were used in the following test. As shown in FIG. 6, consistent with the binding ability to cyno CD16 protein, only 45H6E8 bound efficiently to cyno CD16 expressed on CHO-K1 cells in a dose-dependent manner, with comparable potency to the benchmark antibody P2C47. In contrast, 23H7E4, 37B3G11, 42F5H2 and 91A11D11 showed only weak or marginal binding to cyno CD16. The binding EC₅₀ of tested CD16A chimeric Abs are listed in Table 4.

TABLE 4
Binding activities of the chimeric antibodies to
antigen expressed on cells

| | Antigen | | | |
|---|---|---|---|---|
| | Human | Human | Human | Cyno- |
| | CD16A- | CD16A- | CD16B- | molgus |
| | 158V | 158F | NA1 | CD16 |
| Antibody | EC50 (nM) | | | |
| 23H7E4 | 0.80 | 0.77 | — | — |
| 37B3G11 | 0.68 | 0.74 | — | — |
| 42F5H2 | 0.47 | 0.50 | — | — |
| 45H6E8 | 0.36 | 0.40 | — | 1.74 |
| 91A11D11 | 0.45 | 0.46 | — | — |
| P2C47 | 0.61 | 0.57 | — | 1.74 |

—: No binding

### 2.7. Affinity Measurement

The binding affinity of the chimeric antibodies to human CD16A was determined with Biacore™. Briefly, the antibodies were captured with a Protein A chip. Human CD16A-158V-his and CD16A-158F-his protein at 50 nM was injected over captured antibodies for 30 s at a flow rate of 30 μL/min. The antigen was allowed to dissociate for 500 s. The experiment was carried out on a Biacore™ 8K. Data analysis was carried out using Biacore™ 8K evaluation software. The results showed that all the chimeric CD16A antibodies exhibited high and comparable affinity to both human CD16A-158V and CD16A-158F. Among them, 42F5H2 and 91A11D11 exhibited significantly superior binding affinity to both CD16A forms when compared with benchmark P2C47 (Table 5A and 5B). 23H7E4, 37B3G11 and 45H6E8 showed comparable binding affinity to benchmark P2C47 (Table 5A and 5B).

TABLE 5A
Affinity ranking results of chimeric antibodies

| Affinity | Human CD16A-158V | | |
|---|---|---|---|
| Antibody | ka(1/Ms) | Kd(1/s) | KD(M) |
| 23H7E4 | 2.81 × 10⁶ | 1.07 × 10⁻² | 3.81 × 10⁻⁹ |
| 37B3G11 | 2.98 × 10⁶ | 3.77 × 10⁻³ | 1.26 × 10⁻⁹ |
| 42F5H2 | 4.59 × 10⁶ | 1.39 × 10⁻³ | 3.03 × 10⁻¹⁰ |
| 45H6E8 | 3.52 × 10⁶ | 6.55 × 10⁻³ | 1.86 × 10⁻⁹ |
| 91A11D11 | 6.93 × 10⁶ | 1.42 × 10⁻³ | 2.23 × 10⁻¹⁰ |
| P2C47 | 8.04 × 10⁵ | 1.35 × 10⁻³ | 1.68 × 10⁻⁹ |

TABLE 5B
Affinity ranking results of chimeric antibodies

| Affinity | Human CD16A-158F | | |
|---|---|---|---|
| Antibody | ka(1/Ms) | Kd(1/s) | KD(M) |
| 23H7E4 | 3.16 × 10⁶ | 7.72 × 10⁻³ | 2.45 × 10⁻⁹ |
| 37B3G11 | 3.27 × 10⁶ | 5.31 × 10⁻³ | 1.62 × 10⁻⁹ |
| 42F5H2 | 3.86 × 10⁶ | 8.73 × 10⁻⁴ | 2.26 × 10⁻¹⁰ |
| 45H6E8 | 3.90 × 10⁶ | 4.55 × 10⁻³ | 1.17 × 10⁻⁹ |
| 91A11D11 | 7.39 × 10⁶ | 1.02 × 10⁻³ | 1.38 × 10⁻¹⁰ |
| P2C47 | 8.97 × 10⁵ | 2.00 × 10⁻³ | 2.23 × 10⁻⁹ |

### Example 3. Humanization of CD16A Chimeric Antibodies

### 3.1. Humanization Design of CD16A Chimeric Antibodies

Based on the performance of the chimeric antibodies, 45H6E8 was selected for further humanization. The variable regions were selected to perform humanization. Briefly, the amino acid sequences of the VH and VL were aligned with the available database of human Ig gene sequences to identify the overall best-matching human germline Ig gene sequences. Then, the CDRs of heavy chain and light chain of 45H6E8 chimeric antibodies were grafted onto the candidate human germlines. A 3D model of the grafted antibodies was generated by Molecular Operating Environment (MOE) to determine if there were any critical human amino acids in the framework regions which were essential to be back-mutated to the corresponding mouse amino acids to maintain the CDR conformation and function.

For the heavy chain of 45H6E8, the candidate human germline sequences was the IGHV1-24*01 gene. For the light chain of 45H6E8, the candidate human germline sequences was the IGKV1-39*02 gene. T30K, M48I, R67K, V68A, E72A, and A97T were subjected to back-mutation in the heavy chain framework of human germline sequence IGHV1-24*01. S60D and Y87F were subjected to back-mutation in the light chain framework of human germline sequence IGKV1-39*02.

Different combinations of back-mutation sites were selected to generate variable regions of humanized antibodies. The sequences of the variable regions of the heavy chain and light chain of the humanized 45H6E8 were listed in Table 6A. The pairing of the VH and VL for individual humanized antibodies was listed in Table 6B. The variable regions of humanized antibodies were then fused to the constant region of human IgG1 Fc with L234A/L235A/P329G (LALAPG) mutations for humanized antibody production and functional characterization.

TABLE 6A
Sequences of Variable Regions of 45H6E8 Humanized Antibodies
(underlining indicates CDR; bold/italic indicates back mutations)

| | | SEQ |
|---|---|---|
| 45H6E8 | Sequence | ID NO: |
| 45H6E8 VH | EVQLQQSGAELVRSGASVKLSCTASGFNIKDYYMQWVKQRPEQGLEWIG | 7 |
| | WIDPENGDTEYAPKFQGKATMTADTSSNTAYLQLSSLTSEDTAVYYCTT | |
| | GGNYVDWGQGTTLTVSS | |
| 45H6E8 | QVQLVQSGAEVKKPGASVKVSCKVSGYTLTDYYMQWVRQAPGKGLEWMG | 83 |
| HU-VH1-1 | WIDPENGDTEYAPKFQGRVTMTEDTSTDTAYMELSSLRSEDTAVYYCAT | |
| | GGNYVDWGQGTTVTVSS | |
| 45H6E8 | QVQLVQSGAEVKKPGASVKVSCKVSGYTLKDYYMQWVRQAPGKGLEWIG | 84 |
| HU-VH1-5 | WIDPENGDTEYAPKFQGKATMTADTSTDTAYMELSSLRSEDTAVYYCTT | |
| | GGNYVDWGQGTTVTVSS | |
| 45H6E8 | QVQLVQSGAEVKKPGASVKVSCKVSGYTLTDYYMQWVRQAPGKGLEWIG | 85 |
| HU-VH1-6 | WIDPENGDTEYAPKFQGRVTMTADTSTDTAYMELSSLRSEDTAVYYCTT | |
| | GGNYVDWGQGTTVTVSS | |
| 45H6E8 | QVQLVQSGAEVKKPGASVKVSCKVSGYTLTDYYMQWVRQAPGKGLEWMG | 86 |
| HU-VH1-7 | WIDPENGDTEYAPKFQGRATMTADTSTDTAYMELSSLRSEDTAVYYCTT | |
| | GGNYVDWGQGTTVTVSS | |
| 45H6E8 | QVQLVQSGAEVKKPGASVKVSCKVSGYTLTDYYMQWVRQAPGKGLEWMG | 87 |
| HU-VH1-8 | WIDPENGDTEYAPKFQGRVTMTADTSTDTAYMELSSLRSEDTAVYYCTT | |
| | GGNYVDWGQGTTVTVSS | |
| IGHV1-24*01 | QVQLVQSGAEVKKPGASVKVSCKVSGYTLTELSMHWVRQAPGKGLEWMG | 88 |
| | GFDPEDGETIYAQKFQGRVTMTEDTSTDTAYMELSSLRSEDTAVYYCAT | |
| 45H6E8 VL | DIVMTQSQKFMSTSVGDRVSITCKASQNVGTAVAWYQQKPGQSPKLLIY | 8 |
| | SASNRYTGVPDRFTGSGSGTDFTLTISNMQSEDLADYFCQQYSSYPLLT | |
| | FGAGTNLELK | |
| 45H6E8 | DIQMTQSPSFLSASVGDRVTITCKASQNVGTAVAWYQQKPGKAPKLLIY | 89 |
| HU-VL1-3 | SASNRYTGVPDRFSGSGSGTDFTLTISSLQPEDFATYFCQQYSSYPLLT | |
| | FGQGTRLEIK | |
| IGKV1-39*02 | DIQMTQSPSFLSASVGDRVTITCRASQSISSYLNWYQQKPGKAPKLLIY | 90 |
| | AASSLQSGVPSRFSGSGSGTDFTLTISSLQPEDFATYYCQCGYSTP | |

TABLE 6B
Pairing of VH and VL for 45H6E8
Humanized Antibodies

| 45H6E8 | HU-VL1-3 | VL |
|---|---|---|
| HU-VH1-1 | z13 | |
| HU-VH1-5 | z17 | |
| HU-VH1-6 | z19 | |
| HU-VH1-7 | z20 | |
| HU-VH1-8 | z21 | |
| VH | | Chimeric |

### 3.2 Binding Properties of the CD16A Humanized Antibodies to CD16-Expressing Cells

Cell binding activity of the CD16A humanized antibodies to human CD16A-158V, CD16A-15SF, CD16B-NAT and cynomolgus CD16 was confirmed in cell-based binding assay with antigen-expressing CHO-KT cells. The assay was performed following the protocol described above. The antibodies binding to the antigen on the cell surface were detected with Alexa Fluor® 647 AffiniPure™ F(ab′)₂ fragment goat anti-human IgG Fcγ fragment specific secondary antibody (Jackson Immuno Research, Cat. No. 109-606-098) at a dilution rate of 1:2000.

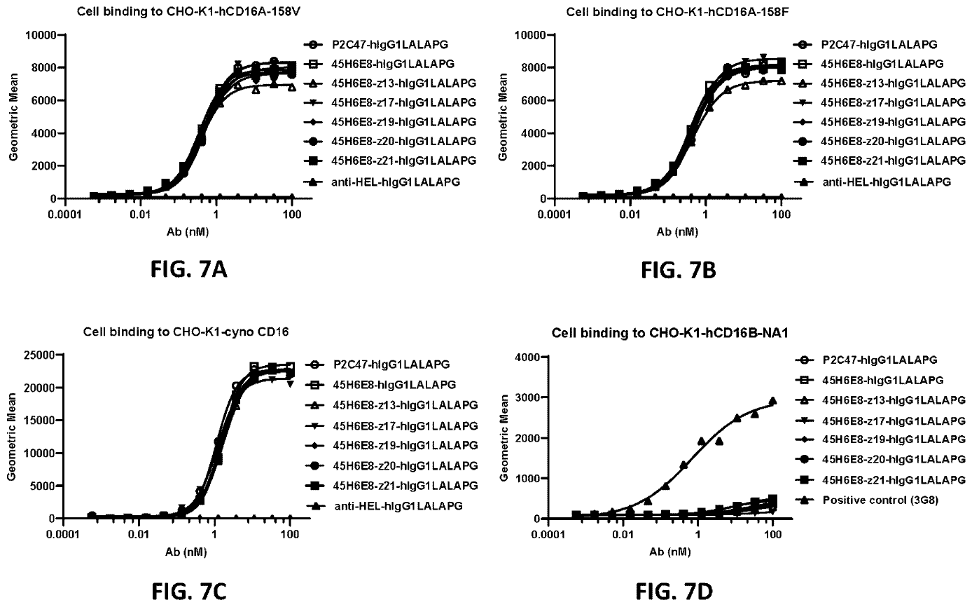

As shown in FIG. 7A and FIG. 7B, all the CD16A humanized antibodies including 45H6E8-z13, 45H6E8-z17, 45H6E8-z19, 45H6E8-z20 and 45H6E8-z21 showed comparable cell binding capability to human CD16A-158V and CD116A-15SF to their chimeric antibody. Meanwhile, these CD16A humanized antibodies displayed similar cell binding ability to cyno CD16 when compared with their chimeric 45H6E8 antibody as shown in FIG. 7C. In contrast, all these CD16A humanized antibodies showed neglectable binding to human CD16B-NA1 expressed on the cells (FIG. 7D).

### Example 4. Optimization of the Humanized CD16A Antibodies

After antibody humanization, CDR regions are often needed to be further optimized to remove potential post-translational modification (PTM) sites in order to increase developability including long-term stability, manufacturability, and homogeneity of the humanized antibodies. PTM such as deamidation, isomerization, glycosylation and oxidation can compromise the potency, efficacy, and safety of therapeutic antibodies.

### 4.1 PTM Removal Design of the Humanized CD16A Antibodies

In this case, computational tools have been used to predict PTM liable sites to facilitate engineering antibodies with better physical and chemical properties. A close examination of the CDR regions of humanized 45H6E8 mAb identified one NG motif located in the CDR2 of the VH region which might undergo deamidation at the Asparagine (N) amino acid and impair the binding and function of the CD16A antibody. Therefore, amino acid replacement of Asparagine to Glutamine (Q) was conducted in this location to remove the potential PTM site. The sequences of the PTM removed variable region of the 45H6E8 humanized mAbs are listed in Table 7A and the mutated CDRs are summarized in Table 7B.

TABLE 7A
Sequences of Variable Regions of CD16A Humanized Antibodies
(underlining indicates CDR; bold/italic indicates PTM mutations)

| | | SEQ |
|---|---|---|
| Name | Sequence | ID NO: |
| 45H6E8-z19 | QVQLVQSGAEVKKPGASVKVSCKVSGYTLTDYYMQWVRQAPGKGLEW | 85 |
| (45H6E8 | IGWIDPENGDTEYAPKFQGRVTMTADTSTDTAYMELSSLRSEDTAVY | |
| HU-VH1-6) VH | YCTTGGNYVDWGQGTTVTVSS | |
| 45H6E8-z19p1 | QVQLVQSGAEVKKPGASVKVSCKVSGYTLTDYYMQWVRQAPGKGLEW | 91 |
| VH | IGWIDPEQGDTEYAPKFQGRVTMTADTSTDTAYMELSSLRSEDTAVY | |
| | YCTTGGNYVDWGQGTTVTVSS | |
| 45H6E8-z20 | QVQLVQSGAEVKKPGASVKVSCKVSGYTLTDYYMQWVRQAPGKGLEW | 92 |
| (45H6E8 | MGWIDPENGDTEYAPKFQGRATMTADTSTDTAYMELSSLRSEDTAVY | |
| HU-VH1-7) VH | YCTTGGNYVDWGQGTTVTVSS | |
| 45H6E8-z20p1 | QVQLVQSGAEVKKPGASVKVSCKVSGYTLTDYYMQWVRQAPGKGLEW | 93 |
| VH | MGWIDPEQGDTEYAPKFQGRATMTADTSTDTAYMELSSLRSEDTAVY | |
| | YCTTGGNYVDWGQGTTVTVSS | |
| 45H6E8-z21 | QVQLVQSGAEVKKPGASVKVSCKVSGYTLTDYYMQWVRQAPGKGLEW | 94 |
| (45H6E8 | MGWIDPENGDTEYAPKFQGRVTMTADTSTDTAYMELSSLRSEDTAVY | |
| HU-VH1-8) VH | YCTTGGNYVDWGQGTTVTVSS | |
| 45H6E8-z21p1 | QVQLVQSGAEVKKPGASVKVSCKVSGYTLTDYYMQWVRQAPGKGLEW | 95 |
| VH | MGWIDPEQGDTEYAPKFQGRVTMTADTSTDTAYMELSSLRSEDTAVY | |
| | YCTTGGNYVDWGQGTTVTVSS | |
| 45H6E8-z19/ | DIQMTQSPSFLSASVGDRVTITCKASQNVGTAVAWYQQKPGKAPKLL | 89 |
| 20/21(p1) | IYSASNRYTGVPDRFSGSGSGTDFTLTISSLQPEDFATYFCQQYSSY | |
| (45H6E8 HU- | PLLTFGQGTRLEIK | |
| VL1-3) VL | | |

TABLE 7B
Optimized Heavy Chain CDR2 of 45H6E8

| Antibody | H-CDR2 | SEQ ID NO: |
|---|---|---|
| 45H6E8-VH | WIDPENGDTEYAPKFQG | 23 |
| 45H6E8-p1 VH | WIDPEQGDTEYAPKFQG | 96 |

### 4.2 Binding Properties of the PTM Removed CD16A Humanized Antibodies to Human CD16-Expressing Cells

Cell binding activity of the PTM removed CD16A humanized antibodies to human CD16A-158V, CD16A-158F, CD16B-NA1 and cynomolgus CD16 was confirmed in cell-based binding assay with antigen-expressing CHO-K1 cells. The assay was performed following the protocol described above.

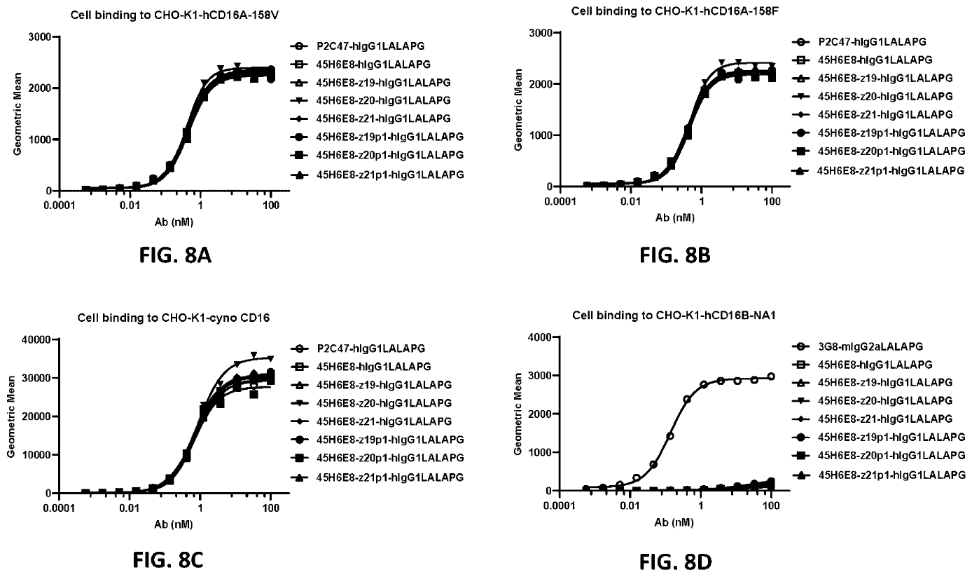

As shown in FIGS. 8A and 8B, all the PTM removed 45H6E8-z19p1, 45H6E8-z20p1 and 45H6E8-z21p1 antibodies retained binding potencies to human CD16A-158V and CD16A-158F expressed on cells as their parental antibodies 45H6E8-z19, 45H6E8-z20 and 45H6E8-z21. Meanwhile, the cell binding efficacy to cyno CD16 of these PTM removed CD16A humanized antibodies was maintained as shown in FIG. 8C. Consistent with their parental humanized antibodies, all these PTM removed mAbs 45H6E8-z19p1, 45H6E8-z20p1 and 45H6E8-z21p1 showed minimal binding to human CD16B-NA1 expressed on the cells (FIG. 8D).

### Example 5. The Binding Ability of CD16A Bi-Specific Fragments

The development strategy of targeting CD16A is to achieve conditional activation of CD16A by the other arms of binding fragments in bi-specific or multiple-specific fragments. Bi-specific or multiple-specific fragments comprised one arm that binds to hCD16A and the other arm(s) that binds to one or more specific antigens. In this case, we initiated to evaluate whether cross-linking of CD16A by tumor associated antigen (TAA)-specific fragments could induce the TAA-dependent conditional activation of CD16A and the resulting functional activation on CD16A expressing effector cells such as NK cells and macrophages.

### 5.1 Generation of Humanized Monoclonal Antibodies Against Human GPC3

As previously descried, GPC3 is a specific TAA for HCC due to its high expression level in tumor cells and limited expression in normal tissues. We have generated a potent humanized monoclonal antibody targeting human GPC3. Briefly, BALB/c mice and C57BL/6 mice were immunized with the human GPC3-his protein. After several rounds of immunization, immune responses were tested by serum ELISA against GPC3-his protein and serum FACS against GPC3 overexpressed CHO-K1 cell line with CHO-K1 parental cell line served as negative control. The resulting mice were used for fusions. The positive hybridoma clones were selected by ELISA and FACS. After subcloning, hybridoma clone 52H5D3B8 was selected, optimized and humanized. The amino acid sequences of the variable region of the heavy chain and light chain of the humanized GPC3 antibody were listed in Table 8 as below.

TABLE 8
Sequences of variable region of humanized
GPC3 antibody (underline indicates CDR)

| | | SEQ |
|---|---|---|
| Name | Sequence | ID NO: |
| Hu 52H5D3B8- | QVQLVQSGAEVKKPGASVKVSCKA | 56 |
| 6#-VH1 | SGYTFTDYEMHWVRQAPGQGLEWM | |
| | GAIHPGSGGTAYNQKFKGRVTMTR | |
| | DTSTSTVYMELSSLRSEDTAVYYC | |
| | ARYYSYAYWGQGTLVTVSS | |
| Hu 52H5D3B8- | DIVMTQTPLSLSVTPGQPASISCR | 57 |
| 6#-VL1 | SSQSLVHSNGNTYLQWYLQKPGQS | |
| | PQLLIYKVSNRFSGVPDRFSGSGS | |
| | GTDFTLKISRVEAEDVGVYYCFQS | |
| | IHVPYTFGGGTKLEIK | |

### 5.2 Construction of CD16A Bi-Specific Fragments

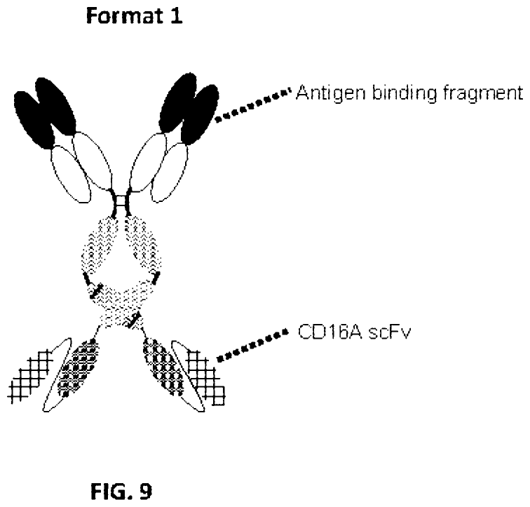

We initiated to explore the possibilities to activate CD16A expressing cells by achieving conditional activation of CD16A through crossing linking by bi-specific fragments. Format 1 (FIG. 9) containing two antigen-binding fragments and two CD16A scFv binding fragments were explored to generate the bi-specific fragments. More specifically, bi-specific fragments were constructed with Format 1 comprising two anti-human CD16A scFv fragments fused to the C-terminus of the ADCC and ADCP disabled Fc fragment of a GPC3-hIgG1LALAPG antibody (SEQ ID NO: 59 and 61 for heavy and light chains, respectively) with a (G4S)4 linker (SEQ ID NO:62). In the scFv of anti-CD16A fragment, the VH and VL were conjugated with a (G4S)3 linker (SEQ ID NO:63). The sequences of heavy chains and the light chains of the GPC3-CD16A bi-specific fragments are listed in Table 9A, Table 9B and Table 9C. GPC3-hIgG1 with wild-type Fc region and GPC3-hIgG1DE mAb carrying ADCC enhanced S239D and 1332E mutation in the Fc region was used as a positive control (SEQ ID NO:58 and 60 for heavy chain, SEQ ID NO: 61 for light chain respectively). GPC3-hIgG1LALAPG mAb carrying LALAPG mutation in the Fc region was used as a negative control (SEQ ID NO: 59 and 61 for heavy and light chains, respectively).

### 5.3 CD16A Bi-Specific Fragments Binding to Human GPC3 Expressed on Cells

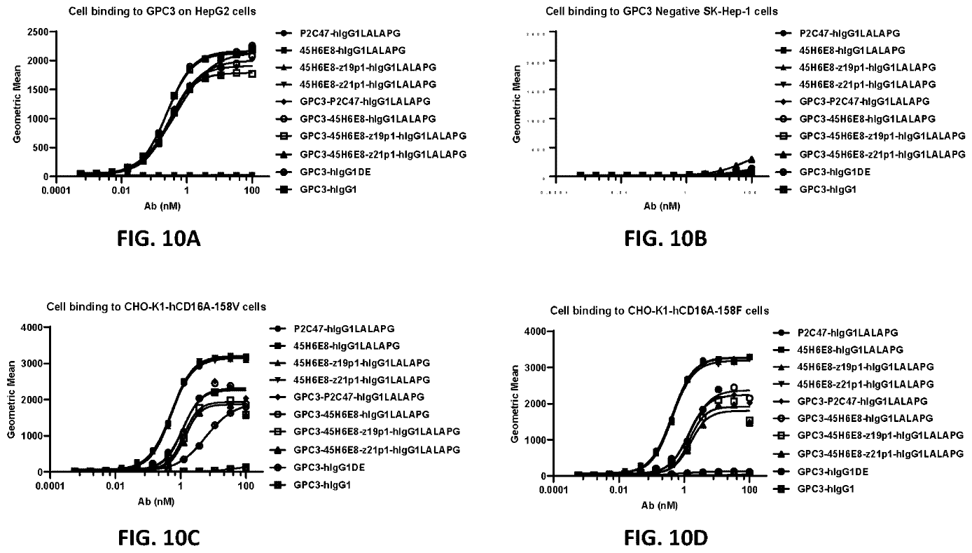

To evaluate the binding activity of GPC3-CD16A bi-specific fragments to GPC3, cell-based binding assay was performed with HepG2 cells, a human hepatocellular carcinoma cell line with high expression level of GPC3, and GPC3-absent SK-HEP-1 cell lines served as a negative control. As shown in FIG. 10A, all the tested GPC3-CD16A bi-specific fragments displayed dose-dependent and specific binding activities to GPC3-positive HepG2 cells, with no detectable binding to GPC3-negative SK-HEP-1 cells (FIG. 10B). More importantly, the binding potency of these bi-specific fragments to GPC3 was almost comparable to that of their parental GPC3 mAbs (FIG. 10A).

### 5.4 CD16A Bi-Specific Fragments Binding to Human CD16A Expressed on Cells

The binding capacity of GPC3-CD16A bi-specific fragments to human CD16A-158V and CD16A-158F expressed on CHO-K1 cells was evaluated by cell-based binding assay. All the tested GPC3-CD16A bi-specific fragments displayed efficient binding potency to both the high-affinity hCD16A-158V and the low-affinity hCD16A-158F expressed on the cells (FIG. 10C and FIG. 10D). A slightly reduced binding ability of bi-specific fragment to CD16A was observed when compared with those of their parental CD16A mAbs, which might result from Fab-scFv format change or steric hindrance induced by C-terminal conjugation of CD16A-scFv fragments. On the contrary, GPC3-hIgG1DE displayed a relatively mild binding capacity to hCD16A-158V expressed on cells although improved binding was observed when compared with GPC3-hIgG1 with poor binding to hCD16A-158V (FIG. 10C). Neither GPC3-hIgG1DE nor GPC3-hIgG1 showed binding to low affinity CD16A-158F (FIG. 10D). Therefore, it is speculated that durable and intensive binding potency of CD16A antibody to both CD16A-158V and CD16A-158F may benefit the long-lasting activation of NK cells and macrophages compared with monoclonal antibodies with ADCC- and ADCP-enhanced engineered Fc region.

TABLE 9A
Sequences of GPC3 mAbs and GPC3-CD16A bi-specific fragments

| | | SEQ |
|---|---|---|
| Name | Sequence | ID NO: |
| Heavy chain of | QVQLVQSGAEVKKPGASVKVSCKASGYTFTDYEMHWVRQAPGQGLEW | 58 |
| GPC3-hIgG1 | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALPAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| Heavy chain of | QVQLVQSGAEVKKPGASVKVSCKASGYTFTDYEMHWVRQAPGQGLEW | 59 |
| GPC3- | MGAIHPGSGGTAYNQKFKGRVTMTRDTSTSTVYMELSSLRSEDTAVY | |
| hIgG1LALAPG | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| Heavy chain of | QVQLVQSGAEVKKPGASVKVSCKASGYTFTDYEMHWVRQAPGQGLEW | 60 |
| GPC3-hIgG1DE | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPDVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALPAPEEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| Light chain of | DIVMTQTPLSLSVTPGQPASISCRSSQSLVHSNGNTYLQWYLQKPGQ | 61 |
| GPC3-hIgG1 | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| (G4S)4 linker | GGGGSGGGGSGGGGSGGGGS | 62 |
| (G4S)3 linker | GGGGSGGGGSGGGGS | 63 |
| 23H7E4 scFv | QVQLQQPGAELVRPGASVKLSCKASGYTFTSYWLNWVKQRPGQGLEW | 64 |
| | IGMIDPSDSETHYNQVFKDKATLTVDKSSTTAYMQLSSLTSEDSAVY | |
| | YCARWMVTKKTFDYWGQGTTLTVSS | |
| | GGGGSGGGGSGGGGS | |
| | DIVLTQSPASLAVSLGQRATISCRASESVEYYGPSLMQWYQQKPGQP | |
| | PKLLIYAASNVESGVPARFSGSGSGTDFSLNIHPVEEDDIAMYFCQQ | |
| | SRKVPYTFGGGAKLEIK | |
| GPC3-23H7E4- | Heavy chain: | 65 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLQQPGAELVRPGASVKLSCKASGYTFTSYWLNWVKQRPGQGLEW | |
| | IGMIDPSDSETHYNQVFKDKATLTVDKSSTTAYMQLSSLTSEDSAVY | |
| | YCARWMVTKKTFDYWGQGTTLTVSS | |
| | GGGGSGGGGSGGGGS | |
| | DIVLTQSPASLAVSLGQRATISCRASESVEYYGPSLMQWYQQKPGQP | |
| | PKLLIYAASNVESGVPARFSGSGSGTDFSLNIHPVEEDDIAMYFCQQ | |
| | SRKVPYTFGGGAKLEIK | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 37B3G11 scFv | QVQLQQPGAELVRPGASVKLSCKASGYTFTTYWMNWVKQRPGQGLEW | 66 |
| | IGMIDPSDSETHYNQMFKDKATLTVDKSSSTAYMQLSSLTSEDSAVY | |
| | YCAVPSLYNSNYRFAYWGQGTLVTVSA | |
| | GGGGSGGGGSGGGGS | |
| | DIVLTQSPASLAVSLGQRATISCRASESVEYYGTSLMQWYQQKPGQP | |
| | PKLLIYSASNVESGVPARFSGSGSGTDFSLNIHPVEEDDIAMYFCQQ | |
| | SRKVPWTFGGGTKLEIK | |
| GPC3-37B3G11- | Heavy chain: | 67 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLQQPGAELVRPGASVKLSCKASGYTFTTYWMNWVKQRPGQGLEW | |
| | IGMIDPSDSETHYNQMFKDKATLTVDKSSSTAYMQLSSLTSEDSAVY | |
| | YCAVPSLYNSNYRFAYWGQGTLVTVSA | |
| | GGGGSGGGGSGGGGS | |
| | DIVLTQSPASLAVSLGQRATISCRASESVEYYGTSLMQWYQQKPGQP | |
| | PKLLIYSASNVESGVPARFSGSGSGTDFSLNIHPVEEDDIAMYFCQQ | |
| | SRKVPWTFGGGTKLEIK | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 42F5H2 scFv | QVQLQQPGAELVRPGASVKLSCKASGYTFTSYWMNWVKQRPGQGLEW | 68 |
| | IGMIDPSDSETHHNQMFKDKATLTVDKSSSTAYMQLSSLTSEDSAVY | |
| | YCAKWLPPYAMDYWGQGTSVTVSS | |
| | GGGGSGGGGSGGGGS | |
| | DIVLTQSPASLAVSLGQRATISCRASESVEYYGPSLMQWYQQKPGQP | |
| | PKLLIYAASNVESGVPARFSGSGSGTDFSLNIHPVEEDDIAMYFCQQ | |
| | SRKVPYTFGGGTKLEIK | |
| GPC3-42F5H2- | Heavy chain: | 69 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLQQPGAELVRPGASVKLSCKASGYTFTSYWMNWVKQRPGQGLEW | |
| | IGMIDPSDSETHHNQMFKDKATLTVDKSSSTAYMQLSSLTSEDSAVY | |
| | YCAKWLPPYAMDYWGQGTSVTVSS | |
| | GGGGSGGGGSGGGGS | |
| | DIVLTQSPASLAVSLGQRATISCRASESVEYYGPSLMQWYQQKPGQP | |
| | PKLLIYAASNVESGVPARFSGSGSGTDFSLNIHPVEEDDIAMYFCQQ | |
| | SRKVPYTFGGGTKLEIK | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8 scFv | EVQLQQSGAELVRSGASVKLSCTASGFNIKDYYMQWVKQRPEQGLEW | 70 |
| | IGWIDPENGDTEYAPKFQGKATMTADTSSNTAYLQLSSLTSEDTAVY | |
| | YCTTGGNYVDWGQGTTLTVSS | |
| | GGGGSGGGGSGGGGS | |
| | DIVMTQSQKFMSTSVGDRVSITCKASQNVGTAVAWYQQKPGQSPKLL | |
| | IYSASNRYTGVPDRFTGSGSGTDFTLTISNMQSEDLADYFCQQYSSY | |
| | PLLTFGAGTNLELK | |
| GPC3-45H6E8- | Heavy chain: | 71 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | EVQLQQSGAELVRSGASVKLSCTASGFNIKDYYMQWVKQRPEQGLEW | |
| | IGWIDPENGDTEYAPKFQGKATMTADTSSNTAYLQLSSLTSEDTAVY | |
| | YCTTGGNYVDWGQGTTLTVSS | |
| | GGGGSGGGGSGGGGS | |
| | DIVMTQSQKFMSTSVGDRVSITCKASQNVGTAVAWYQQKPGQSPKLL | |
| | IYSASNRYTGVPDRFTGSGSGTDFTLTISNMQSEDLADYFCQQYSSY | |
| | PLLTFGAGTNLELK | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 91A11D11 scFv | QVQLQQPGAELVRPGTSVKLSCKASGYTFTSYWMNWVKQRPGQGLEW | 72 |
| | IGMIDPSDSETHYNQMFKDKATLTVDESSSTAYMQLSSLTSEDSAVY | |
| | FCARWTLVVAHFFDYWGQGTTLTVSS | |
| | GGGGSGGGGSGGGGS | |
| | DIVLTQSPASLAVSLGQRATISCRASETVEYYGPSLLQWYQQKPGQP | |
| | PKLLIYGASNVESGVPARFSGSGSGTDFSLNIHPVEEDDVAMYFCQQ | |
| | SRKVPYTFGGGTKLEIK | |
| GPC3-91A11D11- | Heavy chain: | 73 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLQQPGAELVRPGTSVKLSCKASGYTFTSYWMNWVKQRPGQGLEW | |
| | IGMIDPSDSETHYNQMFKDKATLTVDESSSTAYMQLSSLTSEDSAVY | |
| | FCARWTLVVAHFFDYWGQGTTLTVSS | |
| | GGGGSGGGGSGGGGS | |
| | DIVLTQSPASLAVSLGQRATISCRASETVEYYGPSLLQWYQQKPGQP | |
| | PKLLIYGASNVESGVPARFSGSGSGTDFSLNIHPVEEDDVAMYFCQQ | |
| | SRKVPYTFGGGTKLEIK | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| P2C47 scFv | QVQLVQSGAEVKKPGESLKVSCKASGYTFTSYYMHWVRQAPGQGLEW | 74 |
| | MGAIEPMYGSTSYAQKFQGRVTMTRDTSTSTVYMELSSLRSEDTAVY | |
| | YCARGSAYYYDFADYWGQGTLVTVSS | |
| | GGGGSGGGGSGGGGS | |
| | SYVLTQPSSVSVAPGQTATISCGGHNIGSKNVHWYQQRPGQSPVLVI | |
| | YQDNKRPSGIPERFSGSNSGNTATLTISGTQAMDEADYYCQVWDNYS | |
| | VLFGGGTKLTVL | |
| GPC3-P2C47- | Heavy chain: | 75 |
| hIgG1LALAPG | QVQLVQSGAEVKKPGASVKVSCKASGYTFTDYEMHWVRQAPGQGLEW | |
| | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGESLKVSCKASGYTFTSYYMHWVRQAPGQGLEW | |
| | MGAIEPMYGSTSYAQKFQGRVTMTRDTSTSTVYMELSSLRSEDTAVY | |
| | YCARGSAYYYDFADYWGQGTLVTVSS | |
| | GGGGSGGGGSGGGGS | |
| | SYVLTQPSSVSVAPGQTATISCGGHNIGSKNVHWYQQRPGQSPVLVI | |
| | YQDNKRPSGIPERFSGSNSGNTATLTISGTQAMDEADYYCQVWDNYS | |
| | VLFGGGTKLTVL | |
| | Light chain: | 61 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| hIgG1 Fc LALAPG | ASTKGPSVFPLAPSSKSTSGGTAALGCLVKDYFPEPVTVSWNSGALT | 76 |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |

TABLE 9B
Sequences of GPC3-CD16A humanized bi-specific fragments

| | | SEQ |
|---|---|---|
| Name | Sequence | ID NO: |
| 45H6E8-z13 scFv | QVQLVQSGAEVKKPGASVKVSCKVSGYTLTDYYMQWVRQAPGKGLEW | 96 |
| | MGWIDPENGDTEYAPKFQGRVTMTEDTSTDTAYMELSSLRSEDTAVY | |
| | YCATGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 97 |
| z13- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | MGWIDPENGDTEYAPKFQGRVTMTEDTSTDTAYMELSSLRSEDTAVY | |
| | YCATGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 98 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8-z17 scFv | QVQLVQSGAEVKKPGASVKVSCKVSGYTLKDYYMQWVRQAPGKGLEW | 99 |
| | IGWIDPENGDTEYAPKFQGKATMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 100 |
| z17- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLKDYYMQWVRQAPGKGLEW | |
| | IGWIDPENGDTEYAPKFQGKATMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 101 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8-z19 scFv | QVQLVQSGAEVKKPGASVKVSCKVSGYTLTDYYMQWVRQAPGKGLEW | 102 |
| | IGWIDPENGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 103 |
| z19- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | IGWIDPENGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 104 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8-z20 scFv | QVQLVQSGAEVKKPGASVKVSCKVSGYTLTDYYMQWVRQAPGKGLEW | 105 |
| | MGWIDPENGDTEYAPKFQGRATMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 106 |
| z20- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | MGWIDPENGDTEYAPKFQGRATMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 107 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8-z21 scFv | QVQLVQSGAEVKKPGASVKVSCKVSGYTLTDYYMQWVRQAPGKGLEW | 108 |
| | MGWIDPENGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 109 |
| z21- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | MGWIDPENGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 110 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |

TABLE 9C
Sequences of GPC3-CD16A humanized
bi-specific fragments after PTM removal

| | | SEQ |
|---|---|---|
| Name | Sequence | ID NO: |
| 45H6E8-z19p1 | QVQLVQSGAEVKKPGASVKVSCKVSGYTLTDYYMQWVRQAPGKGLEW | 111 |
| scFv | IGWIDPEQGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 112 |
| z19p1- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | IGWIDPEQGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 113 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |
| 45H6E8-z21p1 | QVQLVQSGAEVKKPGASVKVSCKVSGYTLTDYYMQWVRQAPGKGLEW | 114 |
| scFv | MGWIDPEQGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| GPC3-45H6E8- | Heavy chain: | 115 |
| z21p1- | QVQLVQSGAEVKKPGASVKVSCKASGYTFTDYEMHWVRQAPGQGLEW | |
| hIgG1LALAPG | MGAIHPGSGGTAYNQKFKGRVTMTRDTSTSTVYMELSSLRSEDTAVY | |
| | YCARYYSYAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPEAAGGPSVFLFPPKPKDTLMISRTPE | |
| | VTCVVVDVSHEDPEVKFNWYVDGVEVHNAKTKPREEQYNSTYRVVSV | |
| | LTVLHQDWLNGKEYKCKVSNKALGAPIEKTISKAKGQPREPQVYTLP | |
| | PSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPG | |
| | K | |
| | GGGGSGGGGSGGGGSGGGGS | |
| | QVQLVQSGAEVKKPGASVKVSCKVSGYTLTDYYMQWVRQAPGKGLEW | |
| | MGWIDPEQGDTEYAPKFQGRVTMTADTSTDTAYMELSSLRSEDTAVY | |
| | YCTTGGNYVDWGQGTTVTVSS | |
| | GGGGSGGGGSGGGGS | |
| | DIQMTQSPSFLSASVGDRVTITCKASQNVGTAVAWYQQKPGKAPKLL | |
| | IYSASNRYTGVPDRFSGSGSGTDFTLTISSLQPEDFATYFCQQYSSY | |
| | PLLTFGQGTRLEIK | |
| | Light chain: | 116 |
| | DIVMTQTPLSLSVTPGQPASISCRSSQSLVHSNGNTYLQWYLQKPGQ | |
| | SPQLLIYKVSNRFSGVPDRFSGSGSGTDFTLKISRVEAEDVGVYYCF | |
| | QSIHVPYTFGGGTKLEIK | |
| | RTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGL | |
| | SSPVTKSFNRGEC | |

### Example 6. CD16A Bi-Specific Fragments Mediated CD16A Agonistic Signaling

In order to evaluate conditional activation of CD16A by CD16A bi-specific fragments, a CD16A signaling reporter assay was set up. In brief, first, full length human CD16A-158V was introduced into a Jurkat-NFAT luciferase reporter cell line constructed previously, in which the expression of a luciferase gene was under the control of the NFAT transcription factor-responsive promoter. The resulting cell line allows us to evaluate activation of CD16A signaling conveniently by examining downstream NFAT signaling. Then, this Jurkat-hCD16A-158V-NFAT reporter cell line was co-cultured with three human hepatocellular carcinoma cell lines including HepG2, Huh-7 and PLC/PRF/5 with high, medium and low expression levels of GPC3 respectively. SK-HEP-1 with absent GPC3 expression was used as a negative control to exclude the GPC3-independent activation of CD16A. The NFAT signaling pathway would be stimulated once CD16A was activated through GPC3-targeting fragment-mediated binding on GPC3 expressing cells. The luciferase-based chemiluminescence could be detected by ONE-Glo™ Luciferase assay system (Promega, Cat. NO. E6110) and Envision multilabel plate readers (PerkinElemer). The graph and statistics analysis were generated with four-parameter nonlinear regression curve fit in Graphpad Prism 9 software.

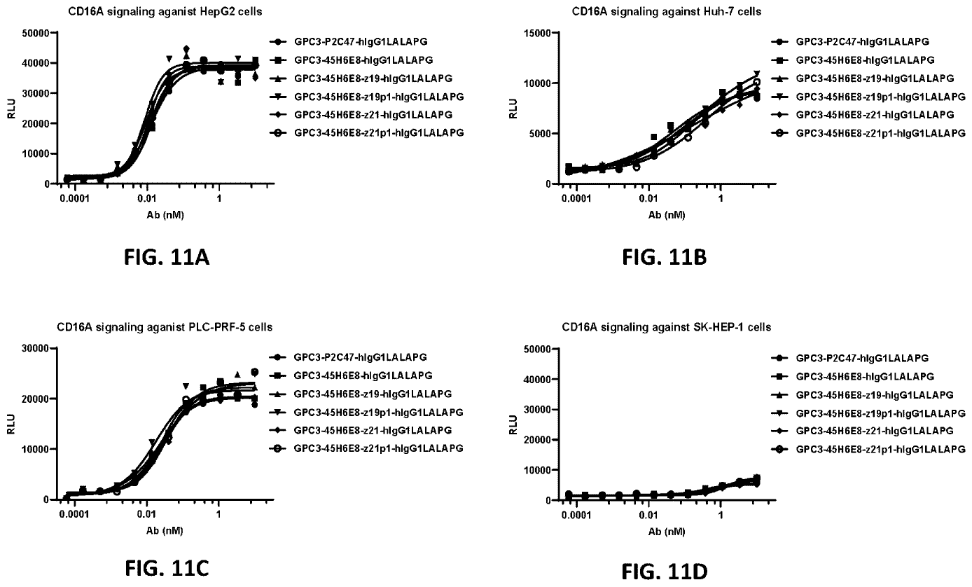

The result showed that all the tested GPC3-CD16A bi-specific fragments efficiently activated CD16A signaling in the presence of GPC3-positive HepG2 (FIG. 11A), Huh7 (FIG. 11B) and PLC/PRF/5 (FIG. 11C) cell lines but not GPC3-negative SK-HEP-1 cell line (FIG. 11D), indicating an efficient and specific GPC3-mediated conditional activation of CD16A signaling by GPC3-CD16A bi-specific fragments even in the presence of moderate or low level of GPC3.

### Example 7. CD16A Bi-Specific Fragments Activate NK Cell Function

In order to evaluate whether conditional activation of CD16A signaling by CD16A bi-specific fragments could translate into activation and cytotoxic effect of NK cells and exhibit functional superiority to Fc-engineered mAb upon target cells engagement, several primary NK cell based functional assays including NK cell cytotoxicity, degranulation and effector cytokine production were performed.

### 7.1 GPC3-CD16A Bi-Specific Fragments Mediated NK Cytotoxicity to Target Cells

To evaluate whether GPC3-mediated CD16A signaling activation could induce a cytotoxic effect of NK cells on target cells, a human primary NK cell-mediated cytotoxicity assay was set-up. Briefly, fresh human primary CD3− CD56+ NK cells were isolated from buffy coat of healthy donors by negative selection with magnetic beads (Miltenyi, Cat. No. 130-092-657). The purity of the isolated NK cells was typically more than 90% monitored by FACS analysis. The NK cells were then either rested in the complete culture media at 37° C. and 5% CO₂ for 24 hours or freshly prepared as effector cells. Three human hepatocellular carcinoma cell lines including HepG2, Huh-7 and PLC/PRF/5 with high, medium and low expression levels of GPC3 respectively were used as target cells. SK-HEP-1 cells were used as the negative control to exclude non-specific killing by NK cells. The NK cells were co-cultured with 1.0×10⁴ target cells at an effector to target ratio of 10:1 in a 96 well microplate. Antibodies were serially diluted at 4-fold starting from 3 nM and added to corresponding wells. After incubation for 4 hours at 37° C., lactate dehydrogenase (LDH) of the supernatant released by damaged cells was measured by LDH cytotoxicity detection kit (Roche, Cat: NO. 04744926001) and detected by Envision multilabel plate readers (PerkinElmer). Formulas for calculating: % cytotoxicity=(LDH release of effector and target cell mix−spontaneous LDH release of effector cells−spontaneous LDH release of untreated target cells)/(maximum LDH release of target cells−spontaneous LDH release of untreated target cells)×100. The graph and statistics analysis were generated with four parameter nonlinear regression curve fit in Graphpad Prism 9 software.

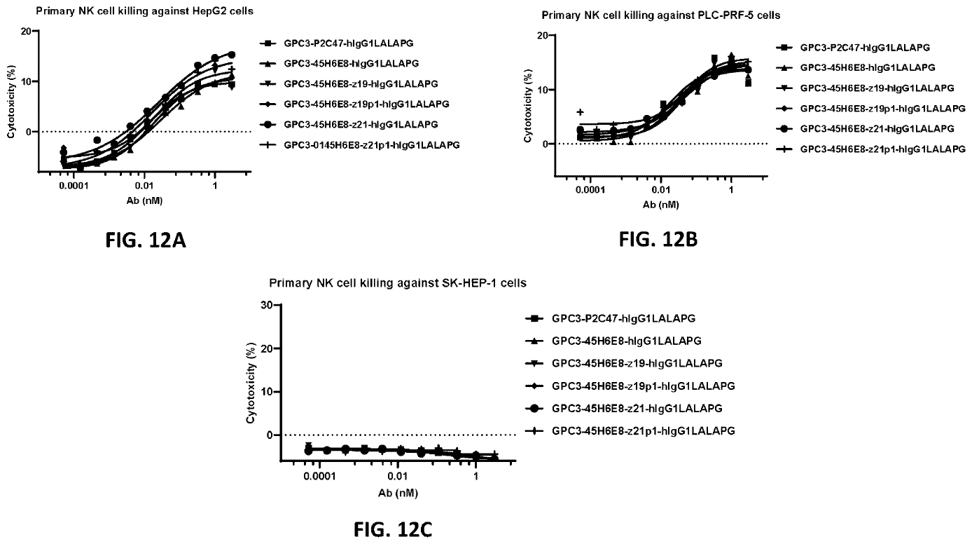

All the tested GPC3-CD16A bi-specific fragments induced NK cell mediated cytotoxicity to GPC3+ target cells HepG2 (FIG. 12A) and Huh-7 (FIG. 12B) in a GPC3-dependent and dose-dependent manner, whereas no cytotoxicity to GPC3 negative SK-HEP-1 cells was observed (FIG. 12C).

### 7.2 GPC3-CD16A Bi-Specific Fragments Stimulated Degranulation of NK Cells

To investigate the ability of the GPC3-CD16A bi-specific fragments to stimulate degranulation of NK cells, expression level of CD107a, a degranulation marker, was analyzed in NK cells. Briefly, human primary NK cells generated as described above were co-incubated with 1.0×10⁵ target cells including HepG2, Huh7 and SK-HEP-1 at E/T ratio of 1:1 in 96-well microplate. Antibodies were serially diluted at 3-fold starting from 10 nM and added into corresponding wells. After 4 hours incubation at 37° C., the mixed cells were harvested and washed to adjust to a concentration of 1×10⁶ cells/mL in ice-cold staining buffer (2% BSA in DPBS). After being pretreated with the Fc blocker, the cells were stained with PE-conjugated human CD107a antibody (2 μL per test, Biolegend, Cat. No. 328608) for 30 mins at 4° C. in the dark. The samples were then fixed with 4% paraformaldehyde and analyzed by flow cytometer LSRFortessa Cell Analyzer (BD Biosciences). Data were analyzed with Flowjo 10.0 software. The graph and statistics analysis were generated with four-parameter nonlinear regression curve fit in Graphpad Prism 9 software.

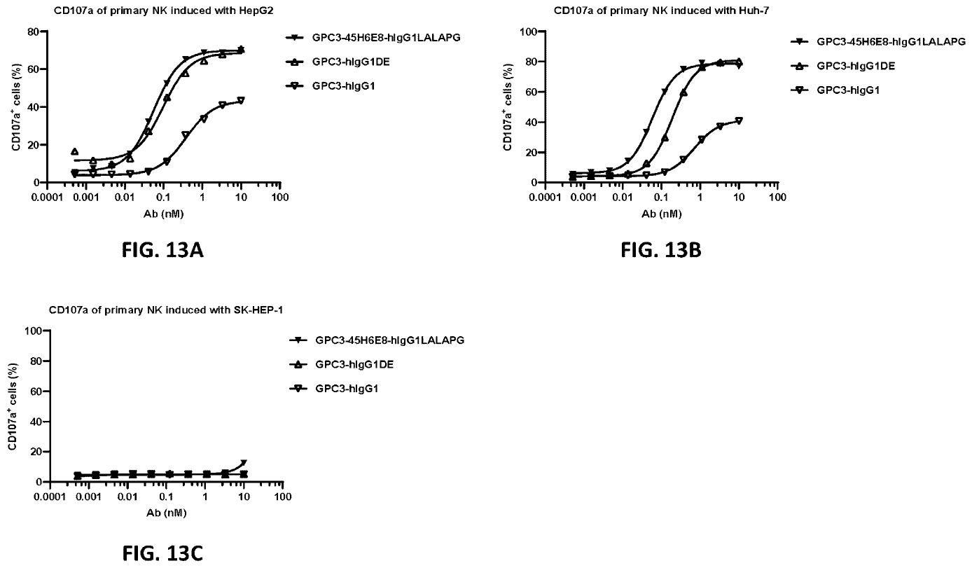

The result showed that GPC3-CD16A bi-specific fragments induced NK cell mediated degranulation as indicated by CD107 upregulation to GPC3+ target cells HepG2 (FIG. 13A) and Huh-7 (FIG. 13B) in a GPC3-dependent and dose-dependent manner, whereas no degranulation of NK cells to GPC3 negative SK-HEP-1 cells was observed (FIG. 13C). More importantly, GPC3-CD16A bi-specific fragments showed enhanced induction of NK cell degranulation when compared with either GPC3-IgG1DE or GPC3-hIgG1 mAbs (FIG. 13A and FIG. 13B), indicating the superiority of the CD16A bi-specific fragments in NK cell activation to the Fc-engineered mAbs.

### 7.3 GPC3-CD16A Bi-Specific Fragments Stimulated Cytokine Production of NK Cells

To investigate the ability of the bi-specific fragments GPC3-CD16A to stimulate the cytokine production of NK cells in the presence of target cells, intercellular IFN-γ and TNF-α in NK cells were analyzed with FACS staining. Briefly, human primary NK cells generated as effector cells were cocultured with 1.0×10⁵ target cells including HepG2, Huh7 and SK-HEP-1 (E/T Ratio=1:1) in 96-well microplate. The protein trafficking inhibitor Brefeldin A (BFA) was added into the co-culture systems to prevent cytokine secretion to the supernatant during NK cell activation. Antibodies were serially diluted at 3-fold starting from 10 nM and added into corresponding wells. After 4 hours incubation at 37° C., the mixed cells were washed and fixed with 4% paraformaldehyde followed by permeabilized with 1× permeabilization buffer (Invitrogen, Cat. No. 00-8333-56) for 20 mins at RT. The cell pellets were resuspended with staining buffer containing diluted APC mouse anti-human IFN-γ antibody (Biolegend, Cat. No. 506510) and BV421 mouse anti-human TNF-α antibody (BD bioscience, Cat. No. 562783) followed by incubation for 30 min at 4° C. in the dark. The samples were washed, resuspended and analyzed by flow cytometer LSRFortessa Cell Analyzer (BD Biosciences). Data were analyzed with Flowjo 10.0 software. The graph and statistics analysis were generated with four-parameter nonlinear regression curve fit in Graphpad Prism 9 software.

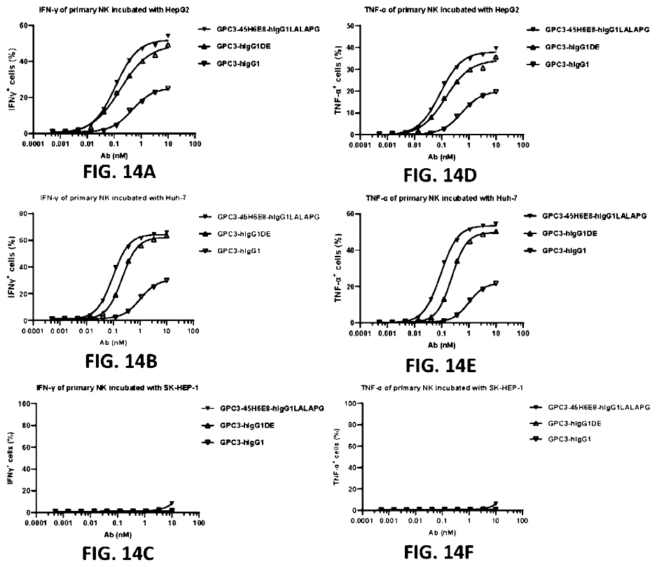

The result showed that GPC3-CD16A bi-specific fragments induced NK cell mediated cytokine production as indicated by the intercellular IFN-γ and TNF-α upregulation to GPC3+ target cells HepG2 (FIG. 14A and FIG. 14D) and Huh-7 (FIG. 14B and FIG. 14E) in a GPC3-dependent and dose-dependent manner, whereas no cytokine production of NK cells to GPC3 negative SK-HEP-1 cells was observed (FIG. 14C and FIG. 14F). More importantly, GPC3-CD16A bi-specific fragments showed enhanced induction of NK cell cytokine release when compared with either GPC3-IgG1DE or GPC3-hIgG1 mAbs (FIG. 14A-14D), indicating the superiority of the CD16A bi-specific fragments in NK cell activation to the Fc-engineered mAbs.

### Example 8: In Vitro Properties of CCR8-CD16A Bi-Specific Antibodies

In order to evaluate whether cross-linking of CD16A by other binding fragments could also induce the conditional activation of CD16A down-stream signaling. CCR8, a chemokine receptor that highly expressed on tumor infiltrating Treg cells was selected as the other binding arm of CD16A bi-specific antibodies (BsAbs).

C-C Motif Chemokine Receptor 8 (CCR8) represents another type of tumor associated antigen. CCR8 was recently identified to be solely expressed on a subset of highly suppressive tumor infiltrating regulatory T cells (Tregs) found in multiple cancers, including breast, colorectal, and lung. Meanwhile, high abundance of CCR8+ Tregs is associated with poor prognosis. Therefore, CCR8 is a promising therapeutic target to augment anti-tumor immunity.

The discovery of CCR8 antibody tested in this example was described as follows. BALB/c mice, C57BL/6 mice and SJL mice were immunized with the full length human CCR8 DNA and CHO-K1/HEK293 hCCR8 cell line. After a routine process of hybridoma technology including several rounds of mice immunization and fusion with immortalized with myeloma cells followed by clone characterization, hybridoma clones were selected for the potent binding to human CCR8 (Table 10A).

TABLE 10A
CCR8 antibody sequences
(underlining indicates CDR)

| | | SEQ |
|---|---|---|
| Name | Sequence | ID NO. |
| Anti-CCR8 | EVQLVESGGGLVQPGGSLRLSCAASGFSF | 117 |
| VH | NTYVMNWVRQAPGKGLEWVARIRTKSNNY | |
| | ATFYVDSVKDRFTISRDDSKNSLYLQMNS | |
| | LKTEDTAVYYCVSGTITRLGAGLDYWGQG | |
| | TLVTVSS | |
| Anti-CCR8 | DIVMTQSPLSLPVTPGEPASISCRSSKSL | 118 |
| VL | LHSNANTYLYWYLQKPGQSPQLLIYRMSN | |
| | LASGVPDRFSGSGSGTAFTLKISRVEAED | |
| | VGVYYCMQHLEYPFTFGGGTKLEIK | |

### 8.1 Construction of CCR8-CD16A Bi-Specific Antibodies

The CCR8-CD16A-hIgG1LALAPG bispecific antibodies were constructed with Format 1 (FIG. 9) comprising two anti-human CD16A scFv fragments fused to the C-terminus of the ADCC disabled Fc fragment of CCR8-hIgG1 antibody. To evaluate whether wildtype Fc would synergize with CD16A-specific binding fragment to contribute to differentiated binding and functional activities, we also generated CCR8-CD16A-hIgG1 bi-specific antibodies with wildtype Fc fragment. In comparison with ADCC enhanced monospecific antibodies (eADCC CCR8 mAb), we produced anti-hCCR8 monoclonal antibodies with Fc-DLE (S239D/A330L/I332E), Fc-DE (S239D/I332E), Fc-afucosylated in which the N-glycan residues in the IgG Fc region missing core fucose sugar units, or wildtype Fc hIgG1. All CCR8-CD16A BsAbs and CCR8 mAbs used the same anti-CCR8 Fab sequences. The sequences of the CCR8-CD16A BsAbs and CCR8 mAbs are listed in Table 10B.

TABLE 10B
Sequences of CCR8-CD16A BsAbs and CCR8 mAbs

| | | SEQ |
|---|---|---|
| Name | Sequence | ID NO. |
| CCR8-hIgG1DE | Heavy chain: | 119 |
| | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSS | |
| | ASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPC | |
| | PAPELLGGPDVFLFPPKPKDTLMISRTPEVTCVVVDVSHEDPEVKFNWYVDGVEVH | |
| | NAKTKPREEQYNSTYRVVSVLIVLHQDWLNGKEYKCKVSNKALPAPEEKTISKAKG | |
| | QPREPQVYTLPPSREEMTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |
| | Light chain: | 120 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-hIgG1DLE | Heavy chain: | 121 |
| | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSS | |
| | ASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPC | |
| | PAPELLGGPDVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPLPEEKTISKAKG | |
| | QPREPQVYTLPPSREEMTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |
| | Light chain: | 122 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 123 |
| hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSEVQLQQSGAELVRSGASVKLSCTASGFNIKDYYMQW | |
| | VKQRPEQGLEWIGWIDPENGDTEYAPKFQGKATMTADTSSNTAYLQLSSLTSEDTA | |
| | VYYCTTGGNYVDWGQGTTLTVSSGGGGSGGGGSGGGGSDIVMTQSQKFMSTSVGDR | |
| | VSITCKASQNVGTAVAWYQQKPGQSPKLLIYSASNRYTGVPDRFTGSGSGTDFTLT | |
| | ISNMQSEDLADYFCQQYSSYPLLTFGAGTNLELK | |
| | Light chain: | 124 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 125 |
| z17-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLKDYYMQW | |
| | VRQAPGKGLEWIGWIDPENGDTEYAPKFQGKATMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 126 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 127 |
| z19-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLTDYYMQW | |
| | VRQAPGKGLEWIGWIDPENGDTEYAPKFQGRVTMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 128 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 129 |
| z19p1-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLTDYYMQW | |
| | VRQAPGKGLEWIGWIDPEQGDTEYAPKFQGRVTMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 130 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 131 |
| z20-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLTDYYMQW | |
| | VRQAPGKGLEWMGWIDPENGDTEYAPKFQGRATMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 132 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 133 |
| z20p1-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLTDYYMQW | |
| | VRQAPGKGLEWMGWIDPEQGDTEYAPKFQGRATMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 134 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CCR8-45H6E8- | Heavy chain: | 135 |
| z21-hIgG1 | EVQLVESGGGLVQPGGSLRLSCAASGFSFNTYVMNWVRQAPGKGLEWVARIRTKSN | |
| | NYATFYVDSVKDRFTISRDDSKNSLYLQMNSLKTEDTAVYYCVSGTITRLGAGLDY | |
| | WGQGTLVTVSSASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLS | |
| | LSPGKGGGGSGGGGSGGGGSQVQLVQSGAEVKKPGASVKVSCKVSGYTLTDYYMQW | |
| | VRQAPGKGLEWMGWIDPENGDTEYAPKFQGRVTMTADTSTDTAYMELSSLRSEDTA | |
| | VYYCTTGGNYVDWGQGTTVTVSSGGGGSGGGGSGGGGSDIQMTQSPSFLSASVGDR | |
| | VTITCKASQNVGTAVAWYQQKPGKAPKLLIYSASNRYTGVPDRFSGSGSGTDFTLT | |
| | ISSLQPEDFATYFCQQYSSYPLLTFGQGTRLEIK | |
| | Light chain: | 136 |
| | DIVMTQSPLSLPVTPGEPASISCRSSKSLLHSNANTYLYWYLQKPGQSPQLLIYRM | |
| | SNLASGVPDRFSGSGSGTAFTLKISRVEAEDVGVYYCMQHLEYPFTFGGGTKLEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |

### 8.2 Binding Ability of CCR8-CD16A Bi-Specific Antibodies to CD16A

The CCR8-45H6E8-hIgG1 BsAb, CCR8-45H6E8-hIgG1LALAPG BsAb and eADCC CCR8 mAb were tested in a cell-based binding assay using CHO-K1 cell lines stably expressing different human Fcγ receptors: hCD16A-158V, hCD16A-158F, or hCD32B, respectively. The purpose of the assay was to evaluate the binding of BsAb to each of the hFcγRs.

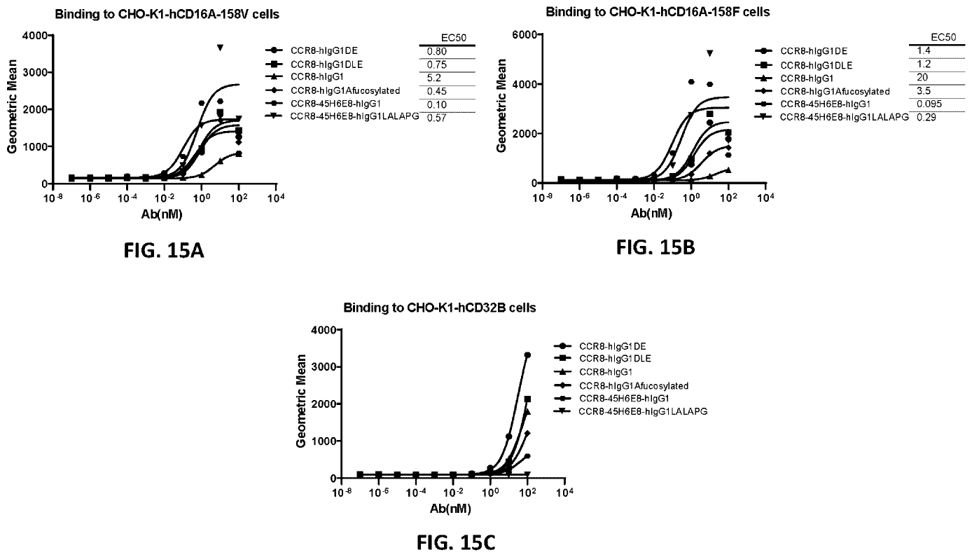

As shown in FIG. 15A and FIG. 15B, CCR8-45H6E8-hIgG1 bsAb strongly bound to both human CD16A-158V and human CD16A-158F in a dose-dependent manner. More importantly, when compared with various eADCC CCR8 mAbs, CCR8-45H6E8-hIgG1 BsAb and CCR8-45H6E8-HIgG1LALAPG BsAb exhibited superior binding potency to both CD16A forms. Of note, CCR8-45H6E8-hIgG1 BsAb displayed a more potent binding ability to human CD16A when compared with CCR8-45H6E8-hIgG1LALAPG BsAb or CCR8-hIgG1 mAb. This phenomenon indicated that wildtype Fc might synergize with CD16A-specific binding fragment to generate an enhanced binding to CD16A. The FIG. 15C indicated CCR8-45H6E8 WT BsAb and CCR8-45H6E8 LALAPG BsAb have the lower binding affinity to the inhibitory Fcγ receptor hCD32B when compared with various CCR8 mAbs. Unexpectedly, when compared with CCR8-hIgG1 mAb, the CCR8-45H6E8-hIgG1 BsAb exhibited a significant reduced binding ability to CD32B.

### 8.3 CCR8-CD16A Bi-Specific Antibodies Stimulated NK Cells Cell Function

To evaluate whether CCR8-CD16A BsAb could induce a cytotoxic effect of NK cells on target cells, degranulation of NK cells and cytokine production of NK cells, a series of functional assay with a human primary NK cell as effector cells and the human T lymphoblast cell line MT-4 naturally expressing human CCR8 as target cells were set-up as described in Example 7.

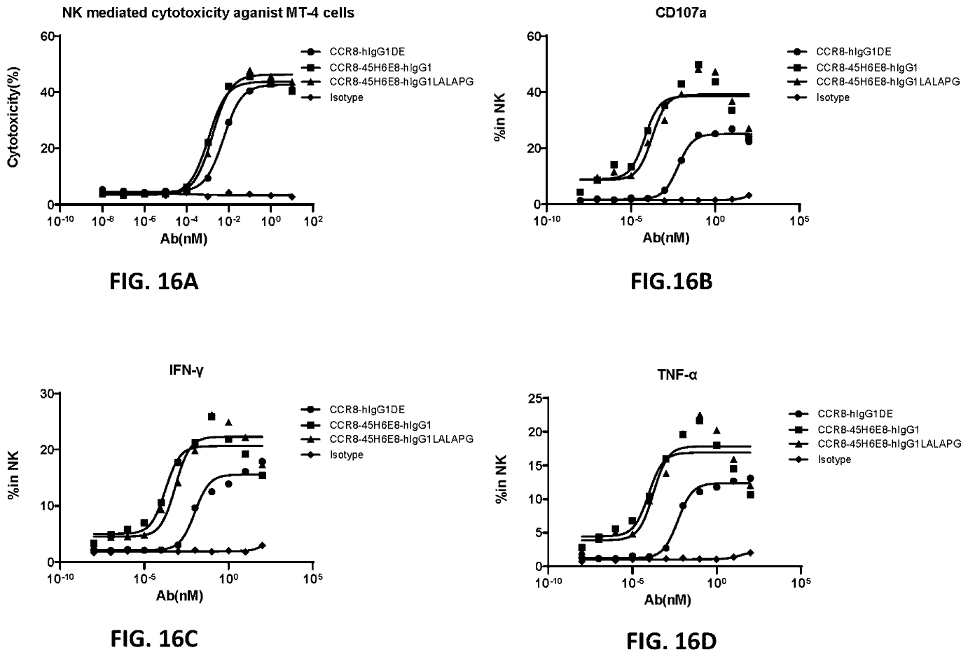

The CCR8-CD16A bi-specific antibodies, both CCR8-45H6E8-hIgG1LALAPG BsAb and CCR8-45H6E8-hIgG1 BsAb, effectively enhanced NK cell function in a dose-dependent and CCR8-dependent manner (FIG. 16A-16D). More importantly, these two CCR8-CD16A BsAbs demonstrated more potent in vitro cytotoxicity against target cells (FIG. 16A), degranulation of NK cells (FIG. 16B), and cytokine production by NK cells (FIGS. 16C and 16D) when compared with ADCC enhanced CCR8-hIgG1DE mAb, indicating the superiority of the CD16A bi-specific fragments in NK cell activation to the Fc-engineered mAbs.

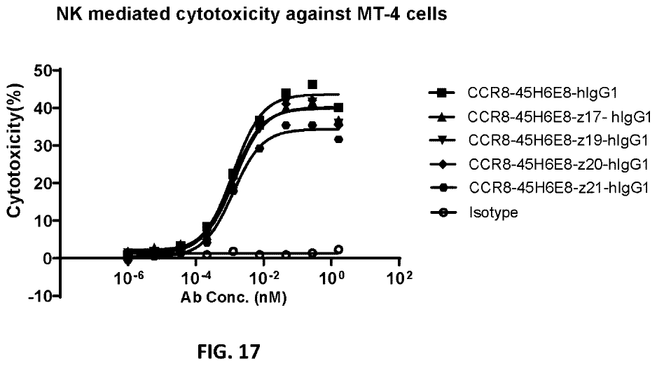

As showed in FIG. 17, humanized CCR8-CD16A BsAb including CCR8-45H6E8-z17-hIgG1, CCR8-45H6E8-z19-hIgG1 and CCR8-45H6E8-z20-hIgG1 showed maintained NK cell mediated killing efficacy compared to the chimeric CCR8-45H6E8-hIgG1 BsAb.

### Example 9: In Vitro Potency of Surrogate CCR8-CD16A BsAb

This example describes the in vitro effect of CCR8-CD16A BsAb and eADCC CCR8 mAb was compared in parallel.

### 9.1 Generation of CCR8 Surrogate Antibody

A surrogate anti-CCR8 antibody 194A1G9 was generated as previously described in Example 8 and selected for its potent binding to human CCR8 while maintaining the cross-reactivity against murine CCR8, which facilitates preclinical efficacy evaluation in murine model. The amino acid sequences of the variable regions of the heavy chain and light chain of the surrogate CCR8 antibodies were listed in Table 11 as below.

TABLE 11
CCR8 antibody sequences
(underlining indicates CDR)

| | | SEQ |
|---|---|---|
| Name | Sequence | ID NO. |
| 194A1G9- | QVQLQQSGTELVRPGASVTLSCK | 77 |
| VH | ASGYTFTDHEMYWVKQTPVHGLE | |
| | WIGAVDPETGSTAYNQKFKVKAI | |
| | LTADKSSSTAYMELRSLTSEDSA | |
| | VYYCTSRLRRAMDNWGHGTSVTV | |
| | SS | |
| 194A1G9- | DVVLTQTPLSLPVSPGDQASISC | 78 |
| VL | RSSQTLLHSSGNTYLHWFLQKPG | |
| | QSPKLLIYKVSNRFSGVPDRFSG | |
| | SGSGTDFTLKISRVEAEDLGVYF | |
| | CSQNTHVPWTFGGGTKLEIK | |

### 9.2 Binding Ability of CCR8 Surrogate Antibody to CCR8

To confirm the binding activity of the surrogate anti-CCR8 antibody 194A1G9, it was subjected to FACS analysis using HEK293 hCCR8 cell line, CHO-K1 mCCR8 cell line and their parental cell lines.

Briefly, HEK293 hCCR8 cell line, CHO-K1 mCCR8 cell line and the parental cell lines were first incubated with serially diluted 194A1G9 at 4° C. for 30 mins. After washing by FACS buffer, PE Goat anti-Human IgG Fc Secondary Antibody (eBioscience™, Invitrogen) was added to each well and incubated at 4° C. for 30 mins. Samples were washed twice with FACS staining buffer. The mean florescence intensity (MFI) of PE was evaluated by MACSQuant Analyzer 16.

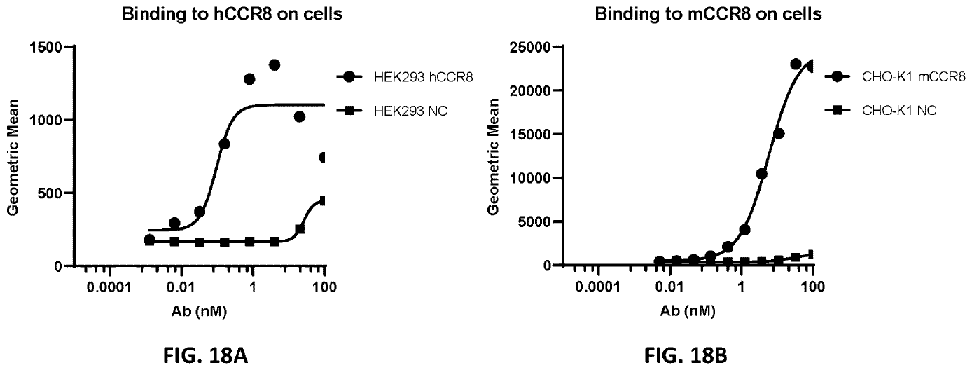

As shown in FIGS. 18A and 18B, 194A1G9 specifically bound to human CCR8 and mCCR8 expressed on cells in a dose-dependent manner.

### 9.3 Construction of Surrogate CCR8-CD16A Bispecific Antibodies

To understand the activity of CCR8-CD16A bispecific antibodies, we generated bi-specific antibodies constructed in Format 1 (FIG. 9) that comprising anti-CD16A scFv fragments 42F51H2 scFv (SEQ ID No. 68) conjugated to the C-terminus of mCCR8-hIgG1 or mCCR8-hIgG1LALA (194A1G9) antibody Fc with a (G4)3 linker (SEQ ID No. 63). The heavy chain and light chain sequence of 194A1G9-42F5H2-hIgG1 BsAb and 194A1G9-42F5H2-hIgG1LALA BsAb were listed in Table 12. For comparison, eADCC mAb 194A1G9 hIgG1 DE was compared in parallel with the sequences of heavy chain and light chain listed in Table 12.

TABLE 12
Sequences of CCR8-CD16A BsAb antibody and CCR8 mAb

| | | SEQ |
|---|---|---|
| Name | Sequence | ID NO. |
| 194A1G9- | Heavy chain: | 79 |
| 42F5H2- | QVQLQQSGTELVRPGASVTLSCKASGYTFTDHEMYWVKQTPVHGLEWIGAVDPET | |
| hIgG1 | GSTAYNQKFKVKAILTADKSSSTAYMELRSLTSEDSAVYYCTSRLRRAMDNWGHG | |
| | TSVTVSS | |
| | ASTKGPSVFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCDKTHTCP | |
| | PCPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| | GGGGSGGGGSGGGGS | |
| | QVQLQQPGAELVRPGASVKLSCKASGYTFTSYWMNWVKQRPGQGLEWIGMIDPSD | |
| | SETHHNQMFKDKATLTVDKSSSTAYMQLSSLTSEDSAVYYCAKWLPPYAMDYWGQ | |
| | GTSVTVSS | |
| | GGGGSGGGGSGGGGS | |
| | DIVLTQSPASLAVSLGQRATISCRASESVEYYGPSLMQWYQQKPGQPPKLLIYAA | |
| | SNVESGVPARFSGSGSGTDFSLNIHPVEEDDIAMYFCQQSRKVPYTFGGGTKLEI | |
| | K | |
| | Light chain: | 80 |
| | DVVLTQTPLSLPVSPGDQASISCRSSQTLLHSSGNTYLHWFLQKPGQSPKLLIYK | |
| | VSNRFSGVPDRFSGSGSGTDFTLKISRVEAEDLGVYFCSQNTHVPWTFGGGTKLE | |
| | IK | |
| | RTVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| 194A1G9- | Heavy chain: | 81 |
| 42F5H2- | QVQLQQSGTELVRPGASVTLSCKASGYTFTDHEMYWVKQTPVHGLEWIGAVDPET | |
| hIgG1LALA | GSTAYNQKFKVKAILTADKSSSTAYMELRSLTSEDSAVYYCTSRLRRAMDNWGHG | |
| | TSVTVSS | |
| | ASTKGPSVFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCDKTHTCP | |
| | PCPAPEAAGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| | GGGGSGGGGSGGGGS | |
| | QVQLQQPGAELVRPGASVKLSCKASGYTFTSYWMNWVKQRPGQGLEWIGMIDPSD | |
| | SETHHNQMFKDKATLTVDKSSSTAYMQLSSLTSEDSAVYYCAKWLPPYAMDYWGQ | |
| | GTSVTVSS | |
| | GGGGSGGGGSGGGGS | |
| | DIVLTQSPASLAVSLGQRATISCRASESVEYYGPSLMQWYQQKPGQPPKLLIYAA | |
| | SNVESGVPARFSGSGSGTDFSLNIHPVEEDDIAMYFCQQSRKVPYTFGGGTKLEI | |
| | K | |
| | Light chain: | 80 |
| | DVVLTQTPLSLPVSPGDQASISCRSSQTLLHSSGNTYLHWFLQKPGQSPKLLIYK | |
| | VSNRFSGVPDRFSGSGSGTDFTLKISRVEAEDLGVYFCSQNTHVPWTFGGGTKLE | |
| | IK | |
| | RTVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| 194A1G9 | Heavy chain: | 82 |
| hIgG1DE | QVQLQQSGTELVRPGASVTLSCKASGYTFTDHEMYWVKQTPVHGLEWIGAVDPET | |
| | GSTAYNQKFKVKAILTADKSSSTAYMELRSLTSEDSAVYYCTSRLRRAMDNWGHG | |
| | TSVTVSS | |
| | ASTKGPSVFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCDKTHTCP | |
| | PCPAPELLGGPDVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPEEKTIS | |
| | KAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| | Light chain: | 80 |
| | DVVLTQTPLSLPVSPGDQASISCRSSQTLLHSSGNTYLHWFLQKPGQSPKLLIYK | |
| | VSNRFSGVPDRFSGSGSGTDFTLKISRVEAEDLGVYFCSQNTHVPWTFGGGTKLE | |
| | IK | |
| | RTVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |

### 9.4 Surrogate CCR8-CD16A Bispecific Antibodies Mediated Activation of ADCC Signaling

Previously, we have observed an enhanced cell binding to activation FcγR CD16A and reduced cell binding to inhibitory FcγR CD32B of CCR8-CD16A bsAb in Example 8. In order to evaluate whether this biased binding profile contribute to improved ADCC signaling, we conducted an in vitro assay to compare the ADCC signaling of these antibodies. In this assay, Jurkat cells were stably co-expressing hCD16A-158V and hCD32B and a luciferase reporter driven by an NFAT-response element (Jurkat-hCD16A-hCD32B-NFAT) as effector cells. Mouse CCR8 expressing CHO-K1 cells (CHO-K1-mCCR8) were used as the target cells. The protocol was as previously described in Example 6.

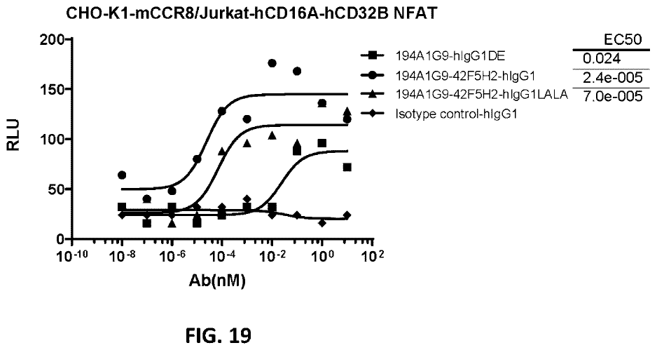

The results suggested that the both 194A1G9-42F5H2-hIgG1 and 194A1G9-421F51H2-hIgG1LALA BsAb could mediate more potent ADCC signaling in comparison with 194A1G9-hIgG1DE in the same experiment setting (FIG. 19). Interestingly, 194A1G9-42F5H2-hIgG1 BsAb showed a superior CD16A activation when compared with 194A1G9-42F5H2-hIgG1LALA BsAb, indicating there existed additive effect from Fc mediated CD16A signaling (FIG. 19).

### Example 10: Synergy Between CD16A-bsAb and Fc Mediated Effector Function In Vivo

To further confirm the function of CD16A BsAb, we used a syngeneic CD16A humanized C57BL/6 mice where the murine FcγRIV gene was replaced by human CD16A 158V to test the in vivo anti-tumor efficacy of the molecules. MC38 cells resuspended in PBS were administered subcutaneously (s.c.) into the right flank of mice at a concentration of 5×10⁵ cells in a volume of 0.1 ml. When the average tumor volume reached approximately 72 mm³, the animals were randomly assigned to experimental groups according to the tumor volume, with 7 animals in each group. Equal molar concentration of surrogate anti-mCCR8 antibodies including 194A1G9-hIgG1DE (6 mg/kg), 194A1G9-42F5H2-hIgG1 BsAb (8 mg/kg) and 194A1G9-42F5-hIgG1LALA BsAb (8 mg/kg) were administered twice every week by intraperitoneal injection. Body weight and tumor volume were measured twice a week.

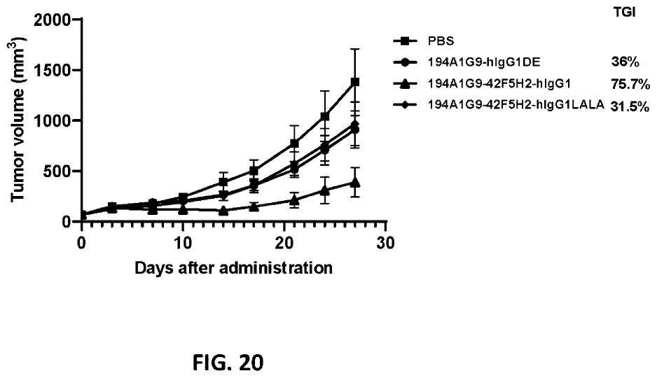

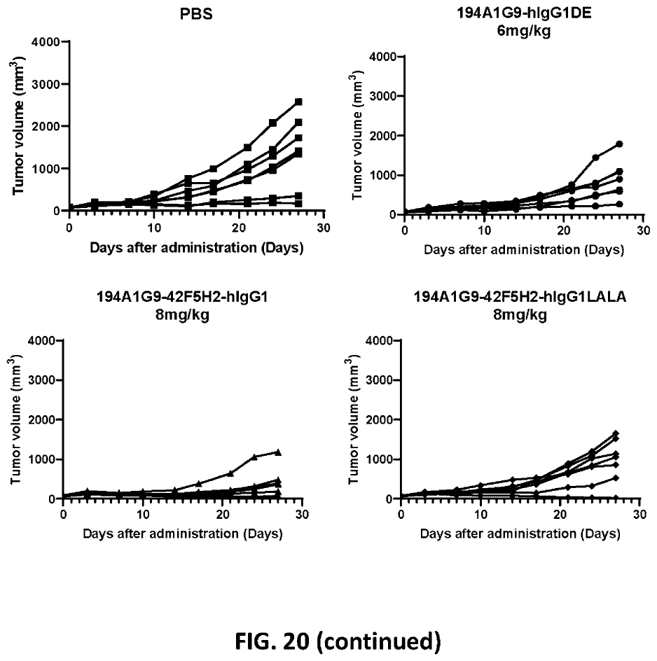

As shown in FIG. 20, compared with the PBS control, 194A1G9-42F5H2-hIgG1LALA BsAb showed comparable inhibition of tumor growth to ADCC enhanced 194A1G9-hIgG1DE mAb, with inhibition rates of 31.5% and 36%, respectively. These data demonstrated that 194A1G9-42F5H2-hIgG1LALA BsAb could efficiently exhibit CCR8-dependent CD16A activation and mediate effector cell mediated depletion of CCR8+ Treg cells and tumor control. Strikingly, 194A1G9-42F5H2-hIgG1 BsAb exhibited a significant improved inhibition of tumor growth with an inhibition rate of 75.7% when compared with 194A1G9-42F5H2-hIgG1LALA BsAb and 194A1G9-hIgG1DE mAb (FIG. 20), indicating a significant contribution of synergy between CD16A antibody mediated and Fc mediated effector function.

In another independent study with the same mouse model, we further confirmed the anti-tumor effect of CD16A BsAb mediated by expression of TAA, such as CCR8 in this example. We used another mCCR8 clone SA214G2 (Biolegend) and another CD16A clone 45H6E8 to generate CCR8-CD16A bispecific antibodies with Format 1 (FIG. 9). Two Fc engineered mAbs SA214G2-hIgG1DE and SA214G2-hIgG1DLE were compared in parallel.

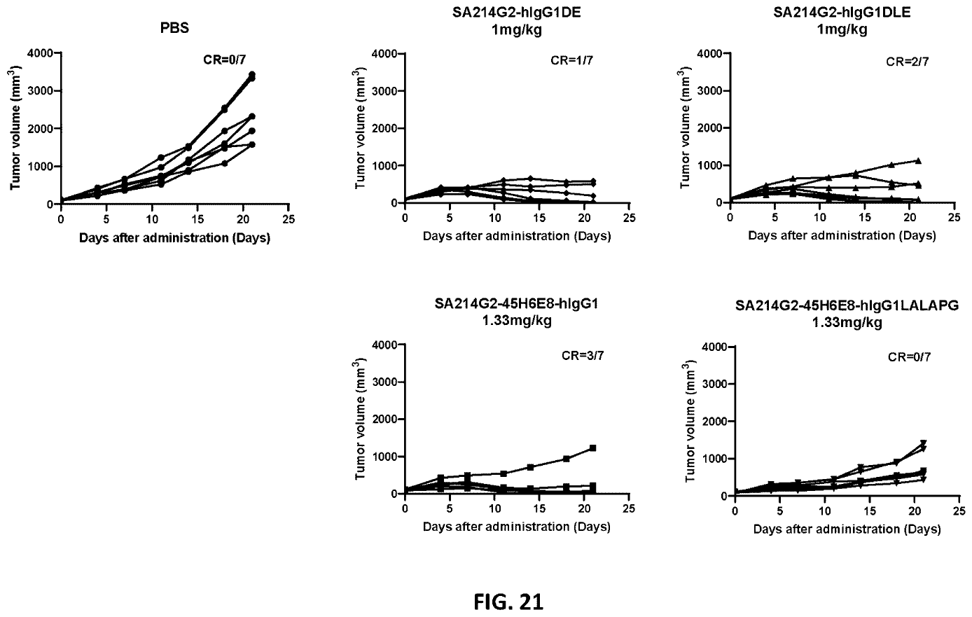

As showed in FIG. 21, compared with the PBS control, SA214G2-45H6E8-hIgG1LALAPG BsAb could efficiently inhibit tumor growth, even at a very low treatment dose (1.33 mg/kg), indicating an efficient activation of effector cell and inhibition of tumor growth solely mediated by CCR8-dependent CD16A activation in the absence of Fc-mediated effector function. Of Note, SA214G2-45H6E8-hIgG1 exhibited a significant improved inhibition of tumor growth with more animal free of tumor after 21 days post 1^{st} administration, compared to other treatment (FIG. 21), further confirming that there was synergistic effect between CD16A antibody mediated and Fc mediated effector function.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. An antibody or antigen binding fragment thereof that has binding specificity to the human CD16A protein, comprising a heavy chain variable region (VH) comprising a VH CDR1, VH CDR2, and VH CDR3, and a light chain variable region (VL) comprising a VL CDR1, VL CDR2 and VL CDR3, wherein: the VH CDR1, VH CDR2, VH CDR3, VL CDR1, VL CDR2, and VL CDR3, respectively, comprise the amino acid sequences of:
(a) SEQ ID NO: 22, 96, 24, 37, 38 and 39;
(b) SEQ ID NO: 22, 23, 24, 37, 38 and 39;
(c) SEQ ID NO: 13, 14, 15, 28, 29 and 30;
(d) SEQ ID NO: 16, 17, 18, 31, 32, and 33;
(e) SEQ ID NO: 19, 20, 21, 34, 35 and 36; or
(f) SEQ ID NO: 25, 26, 27, 40, 41, and 42.

2. (canceled)

3. The antibody or antigen binding fragment thereof of claim 1, wherein the VH CDR1, VH CDR2, VH CDR3, VL CDR1, VL CDR2, and VL CDR3, respectively, comprise the amino acid sequences of SEQ ID NO: 22, 96, 24, 37, 38 and 39.

4. The antibody or antigen binding fragment thereof of claim 3, where the VH comprises an amino acid sequence selected from the group consisting of SEQ ID NO:91, 93 and 95, and the VL comprises an amino acid sequence of SEQ ID NO:89.

5. (canceled)

6. The antibody or antigen binding fragment thereof of claim 1, wherein
(i) the VH comprises an amino acid sequence selected from the group consisting of SEQ ID NO:83-87, and the VL comprises the amino acid sequence of SEQ ID NO:89;
(ii) the VH comprises the amino acid sequence of SEQ ID NO:1, and the VL comprises the amino acid sequence of SEQ ID NO:2;
(iii) the VH comprises the amino acid sequence of SEQ ID NO:3, and the VL comprises the amino acid sequence of SEQ ID NO:4;
(iv) the VH comprises the amino acid sequence of SEQ ID NO:5, and the VL comprises the amino acid sequence of SEQ ID NO:6; or
(v) the VH comprises the amino acid sequence of SEQ ID NO:9, and the VL comprises the amino acid sequence of SEQ ID NO:10.

7-14. (canceled)

15. The antibody or antigen binding fragment thereof of claim 1, further comprising an IgG Fc fragment.

16. (canceled)

17. The antibody or antigen binding fragment thereof of claim 15, wherein the Fc fragment preferably comprises the amino acid sequence of SEQ ID NO:76.

18. A multispecific antibody, comprising:
the antibody or antigen-binding fragment of claim 1;
a second antibody or antigen-binding fragment having binding specificity to a tumor-associated antigen (TAA); and
an Fc fragment that can bind CD64 and can not bind CD16B and/or CD32B;
wherein the antibody or antigen-binding fragment and the Fc fragment have synergism when exerting effector functions.

19-20. (canceled)

21. The multispecific antibody of claim 18, wherein the Fc fragment is a wildtype human IgG Fc fragment, and wherein the Fc fragment is a wildtype human IgG Fc fragment selected from IgG1 Fc, IgG2 Fc, IgG3 Fc and IgG4 Fc.

22-24. (canceled)

25. The multispecific antibody of claim 21, wherein the wildtype Fc fragment is not afucosylated.

26. The multispecific antibody of claim 18, wherein the anti-CD16A antibody or antigen binding fragment thereof and the second antibody or antigen binding fragment thereof are both located N-terminal to the Fc fragment.

27-30. (canceled)

31. The multispecific antibody of claim 18, wherein the TAA is selected from the group consisting of CCR8, CD25, GPC3, Claudin 18.2, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

32. The multispecific antibody of claim 31, wherein the TAA is CCR8, CD25 or GPC3.

33. The multispecific antibody of claim 31, wherein:
(i) the second antibody or antigen binding fragment thereof comprises a heavy chain variable region (VH) comprising the amino acid sequence of SEQ ID NO:56, and a light chain variable region (VL) comprising the amino acid sequence of SEQ ID NO:57;
(ii) the second antibody or antigen binding fragment thereof comprises a heavy chain variable region (VH) comprising the amino acid sequence of SEQ ID NO:117, and a light chain variable region (VL) comprising the amino acid sequence of SEQ ID NO:118; or
(iii) the second antibody or antigen binding fragment thereof comprises a heavy chain variable region (VH) comprising the amino acid sequence of SEQ ID NO:77, and a light chain variable region (VL) comprising the amino acid sequence of SEQ ID NO:78.

34. (canceled)

35. The multispecific antibody of claim 31, which comprises a heavy chain comprising the amino acid sequence of SEQ ID NO:129 and a light chain comprising the amino acid sequence of SEQ ID NO:130, or comprises a heavy chain comprising the amino acid sequence of SEQ ID NO:131 and a light chain comprising the amino acid sequence of SEQ ID NO:132.

36. (canceled)

37. One or more polynucleotide(s) encoding the antibody or antigen binding fragment of claim 1.

38. A cell comprising the one or more polynucleotide(s) of claim 37.

39. (canceled)

40. A method for treating cancer, comprising administering a cancer patient an effective amount of the antibody or antigen binding fragment of claim 1.

41. (canceled)

42. The method of claim 40, wherein the cancer is a nonimmunogenic tumor, wherein the nonimmunogenic tumor is not infiltrated with T cells or is deficient in T cell filtration or T cell activation.

43. (canceled)

44. The method or use of claim 42, wherein the cancer is resistant to a treatment with an immune checkpoint inhibitor.

45. (canceled)

46. The method of claim 40, wherein the cancer is prostate cancer, pancreatic cancer, leukemia, breast cancer, colorectal cancer, gastric cancer, head and neck cancer, liver cancer, esophageal cancer, cervical cancer, thyroid cancer, lung cancer, bladder cancer, kidney cancer, uterus cancer, or melanoma.


SEQUENCE LISTING (136 sequences)

SEQ ID NO: 1 - protein, 119 aa, synthetic construct
QVQLQQPGAELVRPGASVKLSCKASGYTFTSYWLNWVKQRPGQGLEWIGMIDPSDSETHYNQVFKDKATLTVDKSSTTAYMQLSSLTSEDSAVYYCARWMVTKKTFDYWGQGTTLTVSS

SEQ ID NO: 2 - protein, 111 aa, synthetic construct
DIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGAKLEIK

SEQ ID NO: 3 - protein, 121 aa, synthetic construct
QVQLQQPGAELVRPGASVKLSCKASGYTFTTYWMNWVKQRPGQGLEWIGMIDPSDSETHYNQMFKDKATLTVDKSSSTAYMQLSSLTSEDSAVYYCAVPSLYNSNYRFAYWGQGTLVTVSA

SEQ ID NO: 4 - protein, 111 aa, synthetic construct
DIVLTQSPASLAVSLGQRATISCRASESVEYYGTSLMQWYQQKPGQPPKLLIYSASNVESGVPARFSGSGSGTDFSLNIHPVEEDDIAMYFCQQSRKVPWTFGGGTKLEIK

SEQ ID NO: 5 - protein, 118 aa, synthetic construct
QVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSS

SEQ ID NO: 6 - protein, 111 aa, synthetic construct
DIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIK

SEQ ID NO: 7 - protein, 115 aa, synthetic construct
EVQLQQSGAELVRSGASVKLSCTASGFNIKDYYMQWVKQRPEQGLEWIGWIDPENGDTEYAPKFQGKATMTADTSSNTAYLQLSSLTSEDTAVYYCTTGGNYVDWGQGTTLTVSS

SEQ ID NO: 8 - protein, 108 aa, synthetic construct
DIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLLTFGAGTNLELK

SEQ ID NO: 9 - protein, 120 aa, synthetic construct
QVQLQQPGAELVRPGTSVKLSCKASGYTFTSYWMNWVKQRPGQGLEWIGMIDPSDSETHYNQMFKDKATLTVDESSSTAYMQLSSLTSEDSAVYFCARWTLVVAHFFDYWGQGTTLTVSS

SEQ ID NO: 10 - protein, 111 aa, synthetic construct
DIVLTQSPASLAVSLGQRATISCRASETVEYYGPSLLQWYQQKPGQPPKLLIYGASNVESGVPARFSGSGSGTDFSLNIHPVEEDDVAMYFCQQSRKVPYTFGGGTKLEIK

SEQ ID NO: 11 - protein, 120 aa, synthetic construct
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 12 - protein, 106 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 13 - protein, 5 aa, synthetic construct
SYWLN

SEQ ID NO: 14 - protein, 17 aa, synthetic construct
MIDPSDSETHYNQVFKD

SEQ ID NO: 15 - protein, 10 aa, synthetic construct
WMVTKKTFDY

SEQ ID NO: 16 - protein, 5 aa, synthetic construct
TYWMN

SEQ ID NO: 17 - protein, 17 aa, synthetic construct
MIDPSDSETHYNQMFKD

SEQ ID NO: 18 - protein, 12 aa, synthetic construct
PSLYNSNYRFAY

SEQ ID NO: 19 - protein, 5 aa, synthetic construct
SYWMN

SEQ ID NO: 20 - protein, 17 aa, synthetic construct
MIDPSDSETHHNQMFKD

SEQ ID NO: 21 - protein, 9 aa, synthetic construct
WLPPYAMDY

SEQ ID NO: 22 - protein, 6 aa, synthetic construct
DYYMQD

SEQ ID NO: 23 - protein, 17 aa, synthetic construct
WIDPENGDTEYAPKFQG

SEQ ID NO: 24 - protein, 6 aa, synthetic construct
GGNYVD

SEQ ID NO: 25 - protein, 5 aa, synthetic construct
SYWMN

SEQ ID NO: 26 - protein, 17 aa, synthetic construct
MIDPSDSETHYNQMFKD

SEQ ID NO: 27 - protein, 11 aa, synthetic construct
WTLVVAHFFDY

SEQ ID NO: 28 - protein, 15 aa, synthetic construct
RASESVEYYGPSLMQ

SEQ ID NO: 29 - protein, 7 aa, synthetic construct
AASNVES

SEQ ID NO: 30 - protein, 9 aa, synthetic construct
QQSRKVPYT

SEQ ID NO: 31 - protein, 15 aa, synthetic construct
RASESVEYYGTSLMQ

SEQ ID NO: 32 - protein, 7 aa, synthetic construct
SASNVES

SEQ ID NO: 33 - protein, 9 aa, synthetic construct
QQSRKVPWT

SEQ ID NO: 34 - protein, 15 aa, synthetic construct
RASESVEYYGPSLMQ

SEQ ID NO: 35 - protein, 7 aa, synthetic construct
AASNVES

SEQ ID NO: 36 - protein, 9 aa, synthetic construct
QQSRKVPYT

SEQ ID NO: 37 - protein, 11 aa, synthetic construct
KASQNVGTAVA

SEQ ID NO: 38 - protein, 7 aa, synthetic construct
SASNRYT

SEQ ID NO: 39 - protein, 10 aa, synthetic construct
QQYSSYPLLT

SEQ ID NO: 40 - protein, 15 aa, synthetic construct
RASETVEYYGPSLLQ

SEQ ID NO: 41 - protein, 7 aa, synthetic construct
GASNVES

SEQ ID NO: 42 - protein, 9 aa, synthetic construct
QQSRKVPYT

SEQ ID NO: 43 - protein, 449 aa, synthetic construct
QVQLQQPGAELVRPGASVKLSCKASGYTFTSYWLNWVKQRPGQGLEWIGMIDPSDSETHYNQVFKDKATLTVDKSSTTAYMQLSSLTSEDSAVYYCARWMVTKKTFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 218 aa, synthetic construct
DIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGAKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 451 aa, synthetic construct
QVQLQQPGAELVRPGASVKLSCKASGYTFTTYWMNWVKQRPGQGLEWIGMIDPSDSETHYNQMFKDKATLTVDKSSSTAYMQLSSLTSEDSAVYYCAVPSLYNSNYRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 218 aa, synthetic construct
DIVLTQSPASLAVSLGQRATISCRASESVEYYGTSLMQWYQQKPGQPPKLLIYSASNVESGVPARFSGSGSGTDFSLNIHPVEEDDIAMYFCQQSRKVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 448 aa, synthetic construct
QVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 218 aa, synthetic construct
DIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 445 aa, synthetic construct
EVQLQQSGAELVRSGASVKLSCTASGFNIKDYYMQWVKQRPEQGLEWIGWIDPENGDTEYAPKFQGKATMTADTSSNTAYLQLSSLTSEDTAVYYCTTGGNYVDWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 215 aa, synthetic construct
DIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLLTFGAGTNLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 450 aa, synthetic construct
QVQLQQPGAELVRPGTSVKLSCKASGYTFTSYWMNWVKQRPGQGLEWIGMIDPSDSETHYNQMFKDKATLTVDESSSTAYMQLSSLTSEDSAVYFCARWTLVVAHFFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 218 aa, synthetic construct
DIVLTQSPASLAVSLGQRATISCRASETVEYYGPSLLQWYQQKPGQPPKLLIYGASNVESGVPARFSGSGSGTDFSLNIHPVEEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 450 aa, synthetic construct
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 212 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 55 - protein, 115 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSS

SEQ ID NO: 56 - protein, 112 aa, synthetic construct
DIVMTQTPLSLSVTPGQPASISCRSSQSLVHSNGNTYLQWYLQKPGQSPQLLIYKVSNRFSGVPDRFSGSGSGTDFTLKISRVEAEDVGVYYCFQSIHVPYTFGGGTKLEIK

SEQ ID NO: 57 - protein, 445 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 445 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 445 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
DIVMTQTPLSLSVTPGQPASISCRSSQSLVHSNGNTYLQWYLQKPGQSPQLLIYKVSNRFSGVPDRFSGSGSGTDFTLKISRVEAEDVGVYYCFQSIHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 20 aa, synthetic construct
GGGGSGGGGSGGGGSGGGGS

SEQ ID NO: 62 - protein, 15 aa, synthetic construct
GGGGSGGGGSGGGGS

SEQ ID NO: 63 - protein, 245 aa, synthetic construct
QVQLQQPGAELVRPGASVKLSCKASGYTFTSYWLNWVKQRPGQGLEWIGMIDPSDSETHYNQVFKDKATLTVDKSSTTAYMQLSSLTSEDSAVYYCARWMVTKKTFDYWGQGTTLTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGAKLEIK

SEQ ID NO: 64 - protein, 710 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQQPGAELVRPGASVKLSCKASGYTFTSYWLNWVKQRPGQGLEWIGMIDPSDSETHYNQVFKDKATLTVDKSSTTAYMQLSSLTSEDSAVYYCARWMVTKKTFDYWGQGTTLTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGAKLEIK

SEQ ID NO: 65 - protein, 247 aa, synthetic construct
QVQLQQPGAELVRPGASVKLSCKASGYTFTTYWMNWVKQRPGQGLEWIGMIDPSDSETHYNQMFKDKATLTVDKSSSTAYMQLSSLTSEDSAVYYCAVPSLYNSNYRFAYWGQGTLVTVSAGGGGSGGGGSGGGGSDIVLTQSPASLAVSLGQRATISCRASESVEYYGTSLMQWYQQKPGQPPKLLIYSASNVESGVPARFSGSGSGTDFSLNIHPVEEDDIAMYFCQQSRKVPWTFGGGTKLEIK

SEQ ID NO: 66 - protein, 712 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQQPGAELVRPGASVKLSCKASGYTFTTYWMNWVKQRPGQGLEWIGMIDPSDSETHYNQMFKDKATLTVDKSSSTAYMQLSSLTSEDSAVYYCAVPSLYNSNYRFAYWGQGTLVTVSAGGGGSGGGGSGGGGSDIVLTQSPASLAVSLGQRATISCRASESVEYYGTSLMQWYQQKPGQPPKLLIYSASNVESGVPARFSGSGSGTDFSLNIHPVEEDDIAMYFCQQSRKVPWTFGGGTKLEIK

SEQ ID NO: 67 - protein, 244 aa, synthetic construct
QVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIK

SEQ ID NO: 68 - protein, 709 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIK

SEQ ID NO: 69 - protein, 238 aa, synthetic construct
EVQLQQSGAELVRSGASVKLSCTASGFNIKDYYMQWVKQRPEQGLEWIGWIDPENGDTEYAPKFQGKATMTADTSSNTAYLQLSSLTSEDTAVYYCTTGGNYVDWGQGTTLTVSSGGGGSGGGGSGGGGSDIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLLTFGAGTNLELK

SEQ ID NO: 70 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLQQSGAELVRSGASVKLSCTASGFNIKDYYMQWVKQRPEQGLEWIGWIDPENGDTEYAPKFQGKATMTADTSSNTAYLQLSSLTSEDTAVYYCTTGGNYVDWGQGTTLTVSSGGGGSGGGGSGGGGSDIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLLTFGAGTNLELK

SEQ ID NO: 71 - protein, 246 aa, synthetic construct
QVQLQQPGAELVRPGTSVKLSCKASGYTFTSYWMNWVKQRPGQGLEWIGMIDPSDSETHYNQMFKDKATLTVDESSSTAYMQLSSLTSEDSAVYFCARWTLVVAHFFDYWGQGTTLTVSSGGGGSGGGGSGGGGSDIVLTQSPASLAVSLGQRATISCRASETVEYYGPSLLQWYQQKPGQPPKLLIYGASNVESGVPARFSGSGSGTDFSLNIHPVEEDDVAMYFCQQSRKVPYTFGGGTKLEIK

SEQ ID NO: 72 - protein, 711 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQQPGAELVRPGTSVKLSCKASGYTFTSYWMNWVKQRPGQGLEWIGMIDPSDSETHYNQMFKDKATLTVDESSSTAYMQLSSLTSEDSAVYFCARWTLVVAHFFDYWGQGTTLTVSSGGGGSGGGGSGGGGSDIVLTQSPASLAVSLGQRATISCRASETVEYYGPSLLQWYQQKPGQPPKLLIYGASNVESGVPARFSGSGSGTDFSLNIHPVEEDDVAMYFCQQSRKVPYTFGGGTKLEIK

SEQ ID NO: 73 - protein, 241 aa, synthetic construct
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 74 - protein, 706 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 75 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 117 aa, synthetic construct
QVQLQQSGTELVRPGASVTLSCKASGYTFTDHEMYWVKQTPVHGLEWIGAVDPETGSTAYNQKFKVKAILTADKSSSTAYMELRSLTSEDSAVYYCTSRLRRAMDNWGHGTSVTVSS

SEQ ID NO: 77 - protein, 112 aa, synthetic construct
DVVLTQTPLSLPVSPGDQASISCRSSQTLLHSSGNTYLHWFLQKPGQSPKLLIYKVSNRFSGVPDRFSGSGSGTDFTLKISRVEAEDLGVYFCSQNTHVPWTFGGGTKLEIK

SEQ ID NO: 78 - protein, 706 aa, synthetic construct
QVQLQQSGTELVRPGASVTLSCKASGYTFTDHEMYWVKQTPVHGLEWIGAVDPETGSTAYNQKFKVKAILTADKSSSTAYMELRSLTSEDSAVYYCTSRLRRAMDNWGHGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIK

SEQ ID NO: 79 - protein, 219 aa, synthetic construct
DVVLTQTPLSLPVSPGDQASISCRSSQTLLHSSGNTYLHWFLQKPGQSPKLLIYKVSNRFSGVPDRFSGSGSGTDFTLKISRVEAEDLGVYFCSQNTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 706 aa, synthetic construct
QVQLQQSGTELVRPGASVTLSCKASGYTFTDHEMYWVKQTPVHGLEWIGAVDPETGSTAYNQKFKVKAILTADKSSSTAYMELRSLTSEDSAVYYCTSRLRRAMDNWGHGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQQPGAELVRPGASVKLSCKASGYTFTSYWMNWVKQRPGQGLEWIGMIDPSDSETHHNQMFKDKATLTVDKSSSTAYMQLSSLTSEDSAVYYCAKWLPPYAMDYWGQGTSVTVSSGGGGSGGGGSGGGGSDIVLTQSPASLAVSLGQRATISCRASESVEYYGPSLMQWYQQKPGQPPKLLIYAASNVESGVPARFSGSGSGTDFSLNIHPVEEDDIAMYFCQQSRKVPYTFGGGTKLEIK

SEQ ID NO: 81 - protein, 447 aa, synthetic construct
QVQLQQSGTELVRPGASVTLSCKASGYTFTDHEMYWVKQTPVHGLEWIGAVDPETGSTAYNQKFKVKAILTADKSSSTAYMELRSLTSEDSAVYYCTSRLRRAMDNWGHGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 115 aa, synthetic construct
QVQLVQSGAEVKKPGASVKVSCKVSGYTLTDYYMQWVRQAPGKGLEWMGWIDPENGDTEYAPKFQGRVTMTEDTSTDTAYMELSSLRSEDTAVYYCATGGNYVDWGQGTTVTVSS

SEQ ID NO: 83 - protein, 115 aa, synthetic construct
QVQLVQSGAEVKKPGASVKVSCKVSGYTLKDYYMQWVRQAPGKGLEWIGWIDPENGDTEYAPKFQGKATMTADTSTDTAYMELSSLRSEDTAVYYCTTGGNYVDWGQGTTVTVSS

SEQ ID NO: 84 - protein, 115 aa, synthetic construct
QVQLVQSGAEVKKPGASVKVSCKVSGYTLTDYYMQWVRQAPGKGLEWIGWIDPENGDTEYAPKFQGRVTMTADTSTDTAYMELSSLRSEDTAVYYCTTGGNYVDWGQGTTVTVSS

SEQ ID NO: 85 - protein, 115 aa, synthetic construct
QVQLVQSGAEVKKPGASVKVSCKVSGYTLTDYYMQWVRQAPGKGLEWMGWIDPENGDTEYAPKFQGRATMTADTSTDTAYMELSSLRSEDTAVYYCTTGGNYVDWGQGTTVTVSS

SEQ ID NO: 86 - protein, 115 aa, synthetic construct
QVQLVQSGAEVKKPGASVKVSCKVSGYTLTDYYMQWVRQAPGKGLEWMGWIDPENGDTEYAPKFQGRVTMTADTSTDTAYMELSSLRSEDTAVYYCTTGGNYVDWGQGTTVTVSS

SEQ ID NO: 87 - protein, 98 aa, synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 88 - protein, 108 aa, synthetic construct
DIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 89 - protein, 95 aa, synthetic construct
DIQMTQSPSFLSASVGDRVTITCRASQSISSYLNWYQQKPGKAPKLLIYAASSLQSGVPSRFSGSGSGTDFTLTISSLQPEDFATYYCQCGYSTP

SEQ ID NO: 90 - protein, 115 aa, synthetic construct
QVQLVQSGAEVKKPGASVKVSCKVSGYTLTDYYMQWVRQAPGKGLEWIGWIDPEQGDTEYAPKFQGRVTMTADTSTDTAYMELSSLRSEDTAVYYCTTGGNYVDWGQGTTVTVSS

SEQ ID NO: 91 - protein, 115 aa, synthetic construct; identical to SEQ ID NO: 85

SEQ ID NO: 92 - protein, 115 aa, synthetic construct
QVQLVQSGAEVKKPGASVKVSCKVSGYTLTDYYMQWVRQAPGKGLEWMGWIDPEQGDTEYAPKFQGRATMTADTSTDTAYMELSSLRSEDTAVYYCTTGGNYVDWGQGTTVTVSS

SEQ ID NO: 93 - protein, 115 aa, synthetic construct; identical to SEQ ID NO: 86

SEQ ID NO: 94 - protein, 115 aa, synthetic construct
QVQLVQSGAEVKKPGASVKVSCKVSGYTLTDYYMQWVRQAPGKGLEWMGWIDPEQGDTEYAPKFQGRVTMTADTSTDTAYMELSSLRSEDTAVYYCTTGGNYVDWGQGTTVTVSS

SEQ ID NO: 95 - protein, 17 aa, synthetic construct
WIDPEQGDTEYAPKFQG

SEQ ID NO: 96 - protein, 238 aa, synthetic construct
QVQLVQSGAEVKKPGASVKVSCKVSGYTLTDYYMQWVRQAPGKGLEWMGWIDPENGDTEYAPKFQGRVTMTEDTSTDTAYMELSSLRSEDTAVYYCATGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 97 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWMGWIDPENGDTEYAPKFQGRVTMTEDTSTDTAYMELSSLRSEDTAVYYCATGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 98 - protein, 219 aa, synthetic construct; identical to SEQ ID NO: 60

SEQ ID NO: 99 - protein, 238 aa, synthetic construct
QVQLVQSGAEVKKPGASVKVSCKVSGYTLKDYYMQWVRQAPGKGLEWIGWIDPENGDTEYAPKFQGKATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 100 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLKDYYMQWVRQAPGKGLEWIGWIDPENGDTEYAPKFQGKATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 101 - protein, 219 aa, synthetic construct; identical to SEQ ID NO: 60

SEQ ID NO: 102 - protein, 238 aa, synthetic construct
QVQLVQSGAEVKKPGASVKVSCKVSGYTLTDYYMQWVRQAPGKGLEWIGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 103 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWIGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 104 - protein, 219 aa, synthetic construct; identical to SEQ ID NO: 60

SEQ ID NO: 105 - protein, 238 aa, synthetic construct
QVQLVQSGAEVKKPGASVKVSCKVSGYTLTDYYMQWVRQAPGKGLEWMGWIDPENGDTEYAPKFQGRATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 106 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWMGWIDPENGDTEYAPKFQGRATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 107 - protein, 219 aa, synthetic construct; identical to SEQ ID NO: 60

SEQ ID NO: 108 - protein, 238 aa, synthetic construct
QVQLVQSGAEVKKPGASVKVSCKVSGYTLTDYYMQWVRQAPGKGLEWMGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 109 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWMGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 110 - protein, 219 aa, synthetic construct; identical to SEQ ID NO: 60

SEQ ID NO: 111 - protein, 238 aa, synthetic construct
QVQLVQSGAEVKKPGASVKVSCKVSGYTLTDYYMQWVRQAPGKGLEWIGWIDPEQGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 112 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWIGWIDPEQGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 113 - protein, 219 aa, synthetic construct; identical to SEQ ID NO: 60

SEQ ID NO: 114 - protein, 238 aa, synthetic construct
QVQLVQSGAEVKKPGASVKVSCKVSGYTLTDYYMQWVRQAPGKGLEWMGWIDPEQGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 115 - protein, 703 aa, synthetic construct
QVQLVQSGAEVKKPGASVKVSCKASGYTFTDYEMHWVRQAPGQGLEWMGAIHPGSGGTAYNQKFKGRVTMTRDTSTSTVYMELSSLRSEDTAVYYCARYYSYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKVSGYTLTDYYMQWVRQAPGKGLEWMGWIDPEQGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 116 - protein, 219 aa, synthetic construct; identical to SEQ ID NO: 60

SEQ ID NO: 117 - protein, 123 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSS

SEQ ID NO: 118 - protein, 112 aa, synthetic construct
DIVMTQSPLSLPVTPGEPASISCRSSKSLLHSNANTYLYWYLQKPGQSPQLLIYRMSNLASGVPDRFSGSGSGTAFTLKISRVEAEDVGVYYCMQHLEYPFTFGGGTKLEIK

SEQ ID NO: 119 - protein, 453 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 219 aa, synthetic construct
DIVMTQSPLSLPVTPGEPASISCRSSKSLLHSNANTYLYWYLQKPGQSPQLLIYRMSNLASGVPDRFSGSGSGTAFTLKISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 453 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 219 aa, synthetic construct; identical to SEQ ID NO: 120

SEQ ID NO: 123 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLQQSGAELVRSGASVKLSCTASGFNIKDYYMQWVKQRPEQGLEWIGWIDPENGDTEYAPKFQGKATMTADTSSNTAYLQLSSLTSEDTAVYYCTTGGNYVDWGQGTTLTVSSGGGGSGGGGSGGGGSDIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLLTFGAGTNLELK

SEQ ID NO: 124 - protein, 219 aa, synthetic construct; identical to SEQ ID NO: 120

SEQ ID NO: 125 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLKDYYMQWVRQAPGKGLEWIGWIDPENGDTEYAPKFQGKATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 126 - protein, 219 aa, synthetic construct; identical to SEQ ID NO: 120

SEQ ID NO: 127 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLTDYYMQWVRQAPGKGLEWIGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 128 - protein, 219 aa, synthetic construct; identical to SEQ ID NO: 120

SEQ ID NO: 129 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLTDYYMQWVRQAPGKGLEWIGWIDPEQGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 130 - protein, 219 aa, synthetic construct; identical to SEQ ID NO: 120

SEQ ID NO: 131 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLTDYYMQWVRQAPGKGLEWMGWIDPENGDTEYAPKFQGRATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 132 - protein, 219 aa, synthetic construct; identical to SEQ ID NO: 120

SEQ ID NO: 133 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLTDYYMQWVRQAPGKGLEWMGWIDPEQGDTEYAPKFQGRATMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 134 - protein, 219 aa, synthetic construct; identical to SEQ ID NO: 120

SEQ ID NO: 135 - protein, 706 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGYTLTDYYMQWVRQAPGKGLEWMGWIDPENGDTEYAPKFQGRVTMTADTSTDTAYMELSSLRSEDTAVYYCTTGGNYVDWGQGTTVTVSSGGGGSGGGGSGGGGSDIQMTQSPSFLSASVGDRVTITCKASQNVGTAVAWYQQKPGKAPKLLIYSASNRYTGVPDRFSGSGSGTDFTLTISSLQPEDFATYFCQQYSSYPLLTFGQGTRLEIK

SEQ ID NO: 136 - protein, 219 aa, synthetic construct; identical to SEQ ID NO: 120